(19) United States
(12) Patent Application Publication
(10) Pub. No.: US 2026/0024194 A1
(43) Pub. Date: Jan. 22, 2026

(54) METHODS AND SYSTEMS FOR DETECTION AND CORRECTION OF NON-PHYSIOLOGICAL CARDIAC STRAIN TRACES

(71) Applicant: GE Precision Healthcare LLC, Waukesha, WI (US)

(72) Inventor: Hani Nozari Mirar, Oslo (NO)

(21) Appl. No.: 18/774,835

(22) Filed: Jul. 16, 2024

(51) Int. Cl. G06T 7/00; A61B 8/00; A61B 8/08; G06T 7/11

(52) U.S. Cl. CPC G06T 7/0012; A61B 8/0883; A61B 8/485; G06T 7/11; A61B 8/461; G06T 2207/10132; G06T 2207/30048

(57) ABSTRACT

Systems are herein provided for detection and correction of non-physiological strain traces. In one example, a method comprises generating cardiac ultrasound images from ultrasound imaging data of a heart, generating a segmented region of interest (ROI) of the cardiac ultrasound images, identifying a plurality of points within the segmented ROI, identifying one or more of the plurality of points that correspond to one or more sources of non-physiological strain, correcting the one or more of the plurality of points to generate a corrected segmented ROI with a corrected plurality of points, calculating strain values for the corrected segmented ROI.

### FIELD

Embodiments of the subject matter disclosed herein relate to ultrasound imaging, and more specifically to detection and region of interest-based correction of non-physiological strain traces in cardiac ultrasound imaging.

### BACKGROUND

An ultrasound imaging system typically includes an ultrasound probe that is applied to a patient's body and a workstation or device that is operably coupled to the probe. During a scan, the probe may be controlled by an operator of the system and is configured to transmit and receive ultrasound signals that are processed into an ultrasound image by the workstation or device. The workstation or device may show the ultrasound images as well as a plurality of user-selectable inputs through a display device. The operator or other user may interact with the workstation or device to analyze the images displayed on and/or select from the plurality of user-selectable inputs. As an example, the user may select a region of interest in cardiac ultrasound images from which cardiac strain values may be calculated.

### BRIEF DESCRIPTION

In one example, a system, comprises: an ultrasound probe comprising at least one transducer, a matching layer, and a damping block; a display device; and a processor configured to execute instructions stored in non-transitory memory that, when executed, cause the processor to: acquire ultrasound imaging data of a heart via the ultrasound probe; generate cardiac ultrasound images from the acquired ultrasound imaging data of the heart; generate a region of interest (ROI) comprising a plurality of segments; identify a plurality of speckle points within each of the plurality of segments of the ROI; determine motion vectors of each of the plurality of speckle points within each of the plurality of segments; determine, based on the motion vectors, one or more sources of non-physiological strain corresponding to one or more of the plurality of speckle points; identify a type of the one or more sources of non-physiological strain; in response to identifying the type as ROI-based, correct the ROI to generate a corrected ROI; determine cardiac strain values in the corrected ROI via a speckle tracking algorithm; and output the cardiac strain values on the display device.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

Various aspects of this disclosure may be better understood upon reading the following detailed description and upon reference to the drawings, in which:

FIG. 1 shows a block schematic diagram of an ultrasound imaging system, according to an embodiment;

FIG. 2 is a schematic diagram illustrating an image processing system for analyzing ultrasound images;

FIG. 3 shows a first ultrasound image with a region of interest (ROI) and points thereof;

FIG. 4 shows the ultrasound image and ROI of FIG. 3 with overlaid segments and strain traces thereof;

FIG. 5 shows a first example strain trace plot of the first ultrasound image of FIG. 3;

FIG. 6 shows a second example strain trace plot of the first ultrasound image of FIG. 3;

FIG. 7A shows a second ultrasound image with multiple overlaid segments of an ROI;

FIG. 7B shows polar graphs of points of the segments of the ROI of the second ultrasound image of FIG. 7A;

FIG. 8A shows a third ultrasound image with an overlaid segment of an ROI including two motion clusters;

FIG. 8B shows the third ultrasound image with the overlaid segment and points of the motion clusters;

FIG. 8C shows segments of the third ultrasound image with strain traces thereof;

FIG. 8D shows a corresponding strain trace graph of the third ultrasound image;

FIG. 8E shows a polar graph of the motion vectors of the third ultrasound image;

FIG. 9A shows a fourth ultrasound image with an overlaid segment of an ROI including two motion clusters;

FIG. 9B shows the fourth ultrasound image with the overlaid segment and points of the motion clusters;

FIG. 9C shows segments of the fourth ultrasound image with strain traces thereof and a corresponding strain trace plot;

FIG. 9D shows a corresponding strain trace graph of the fourth ultrasound image;

FIG. 9E shows a polar graph of motion vectors of the fourth ultrasound image;

FIG. 10A shows a fifth ultrasound image with an overlaid segment of an ROI including a noisy motion cluster;

FIG. 10B shows a strain trace plot corresponding to the overlaid segment of the ROI;

FIG. 10C shows a polar graph of motion vectors of the fifth ultrasound image;

FIG. 11A shows a first example of ROI-based correction of non-physiological strain trace of the ROI of the third ultrasound image;

FIG. 11B shows a second example of ROI-based correction of non-physiological strain trace of the ROI of the fourth ultrasound image;

FIG. 12A shows the third ultrasound image and ROI thereof before and after correction;

FIG. 12B shows strain trace plots of the third ultrasound image before and after correction;

FIG. 13A shows the fourth ultrasound image and ROI thereof before and after correction;

FIG. 13B shows strain trace plots of the fourth ultrasound image before and after correction;

FIG. 14 shows a flowchart illustrating a method for ROI-based correction of non-physiological strain traces; and

FIG. 15 shows a flowchart illustrating a method for detecting non-physiological strain traces.

### DETAILED DESCRIPTION

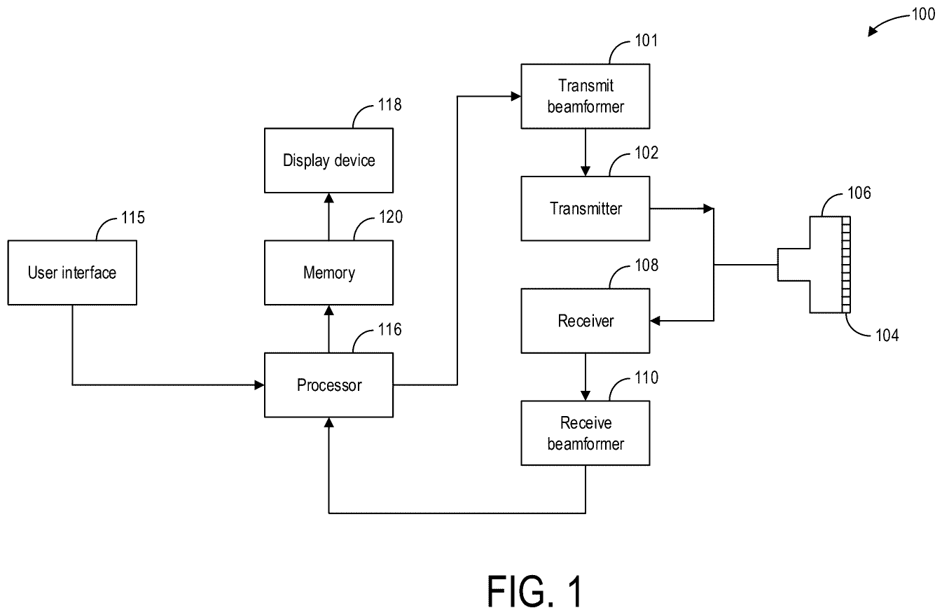

Embodiments of the present disclosure will now be described, by way of example, with reference to the FIGS. 1-15, which relate to various embodiments for cardiac ultrasound imaging. In particular, systems and methods are provided for detecting non-physiological strain traces of cardiac ultrasound imaging and, when the non-physiological strain traces are due to ROI misalignment, performing ROI-based correction of the non-physiological strain traces.

Contraction and relaxation of cardiac muscles during each heartbeat results in longitudinal, circumferential, and radial strain values, which may be calculated via a strain tool used in cardiac ultrasound imaging (e.g., speckle tracking echocardiography). Such cardiac strain values may provide information on regions of the heart with impaired cardiac muscle function. As such, cardiac ultrasound imaging using speckle tracking echocardiography is a useful diagnostic tool.

Speckle tracking echocardiography may include defining a region of interest (ROI) (e.g., either automatically or manually set by a user) and tracking positional changes in brighter intensity pixel areas, known as speckles, over time. However, multiple sources of non-physiological strain exist that affect calculated strain. Firstly, the calculated cardiac strain values may be inaccurate based on the tissue included in the region of interest. For accurate cardiac strain values, it is desired to include only the myocardium, with the endocardium and epicardium layers as the inner-most and outer-most regions, respectively. However, because the distance between the epicardium and the non-contractile pericardium is small, and because the pericardium layer has high image intensities, the pericardium may be erroneously included in the ROI. Since the pericardium is bright but does not contain contractile muscle, the inclusion of the pericardium may result in an underestimation of both global and regional strain values. When enough non-contractile pericardium is included, even a healthy region may be identified as infarcted due to the low strain values indicating impaired heart motion. As a result, additional time and effort may be spent trying to diagnose the patient. Secondly, myocardium may become obscured due to image artifacts such as haze, reverberation, shadowing, or other form of noise. In examples where the ROI includes image artifacts, the strain values and shape of the strain trace may be impacted as the artifacts affect the speckle tracking algorithm. Regions that include noise may result in non-usable strain traces, thus resulting in increased time and effort spent repeating imaging or analyzing the images.

Detection of sources of non-physiological strain is difficult for human users. Specifically, discerning non-physiological strain traces in outputted strain trace graphs can be difficult or all together impossible for the user. Additionally, a human user may not be provided outputs with speckle points, as shown in some of the figures herein, to analyze for motion patterns. Rather speckle tracking algorithms may be applied in the background and a strain trace graph may be outputted for user for diagnostic purposes. Thus, even if a human user does discern an abnormality indicative of non-physiological strain in an outputted strain trace graph, correcting for this may be challenging and may ultimately include repeating segmentation of the ROI and calculation of strain and in some examples even repeating the scan acquisition entirely, which is both time consuming and inefficient to the system.

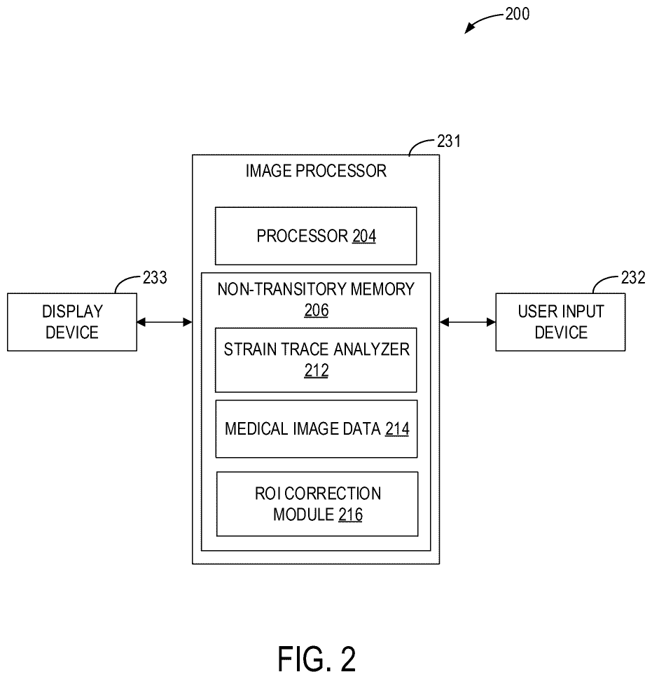

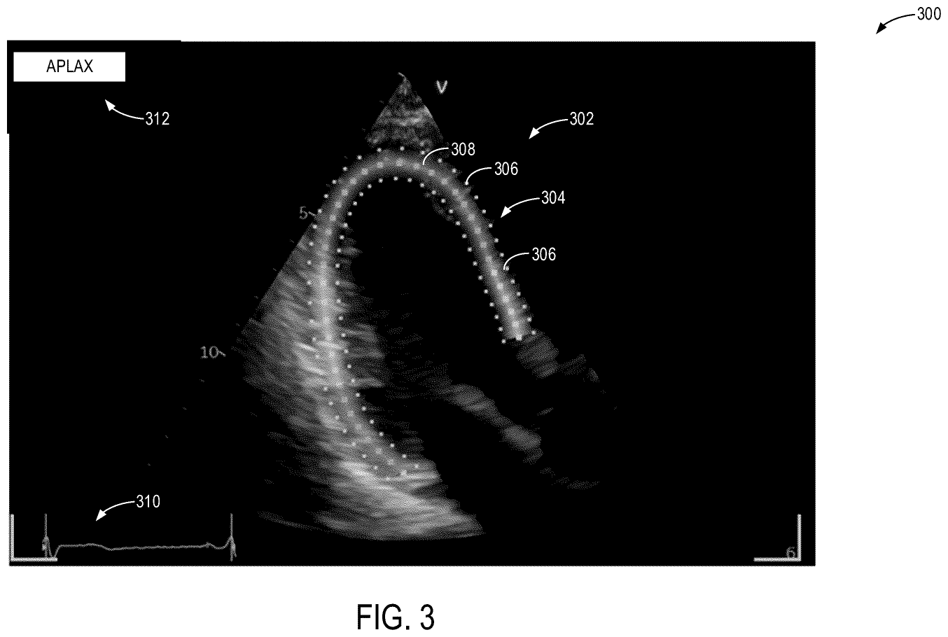

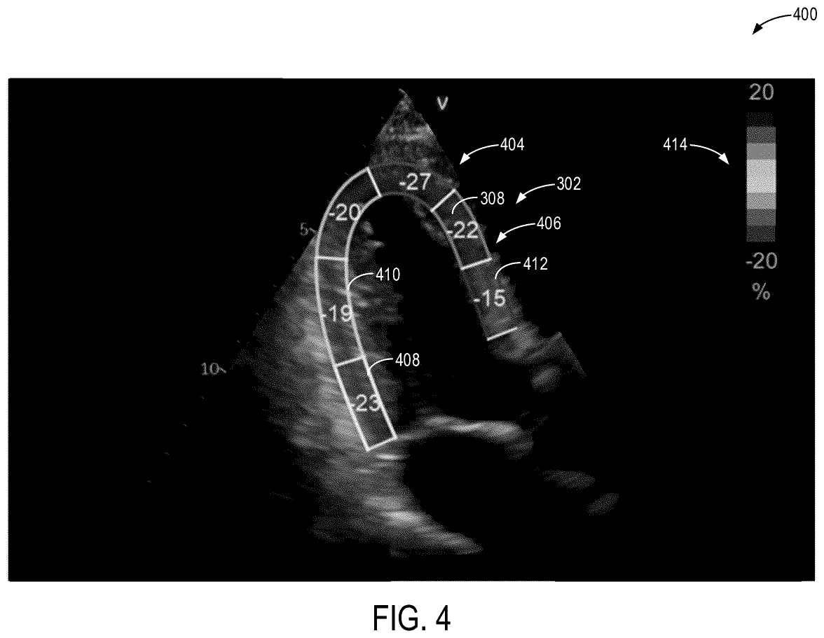

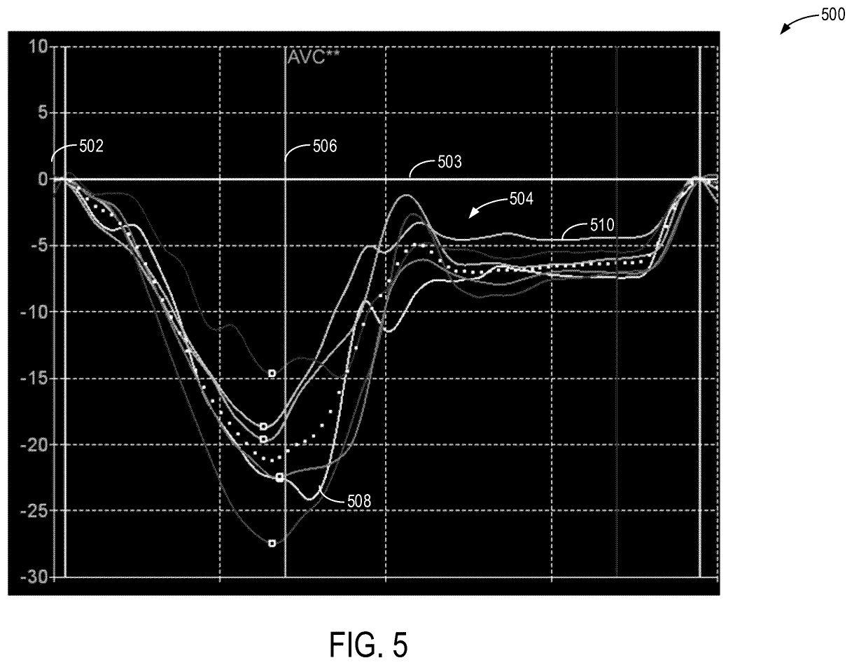

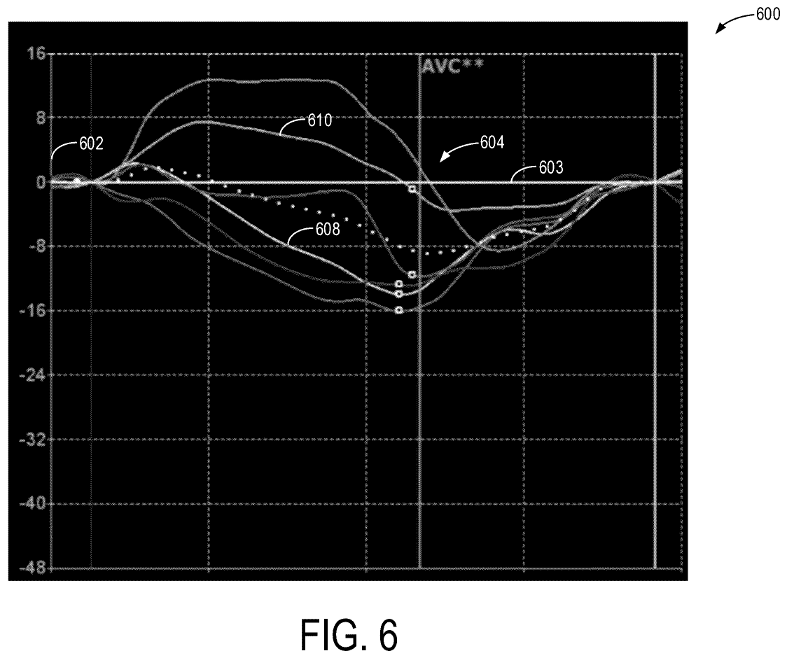

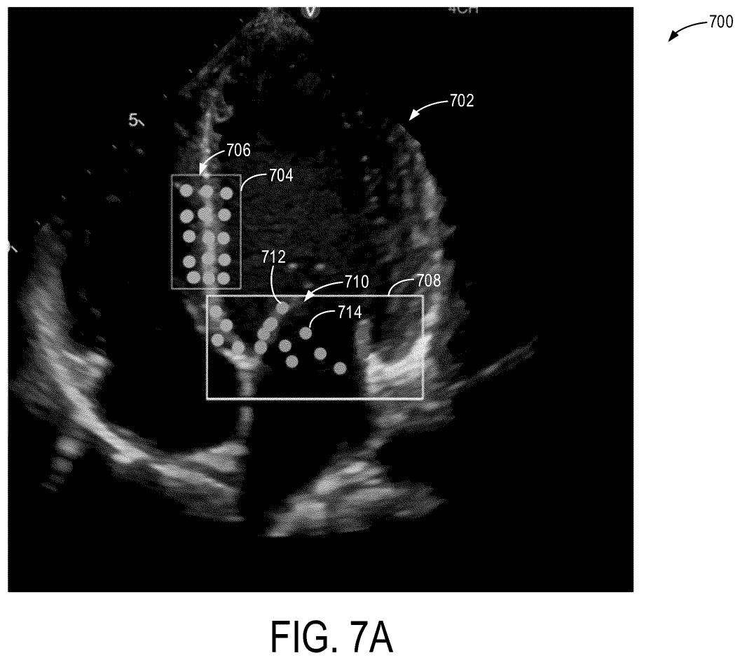

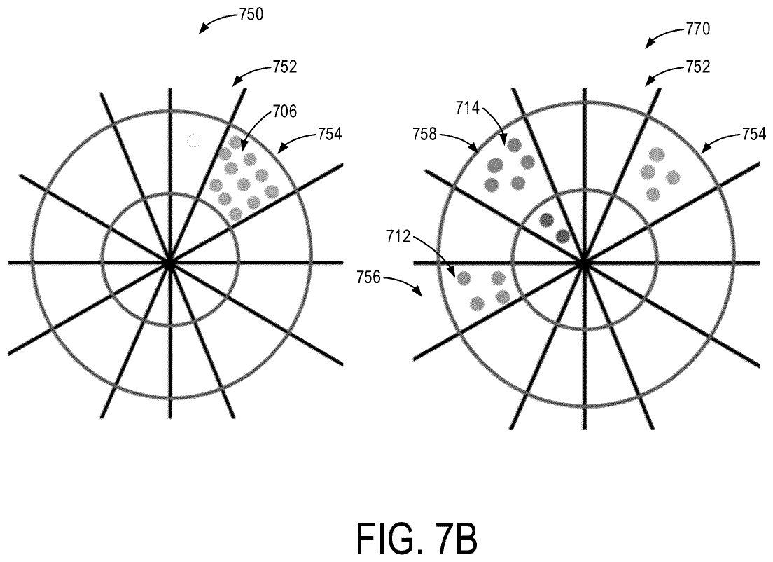

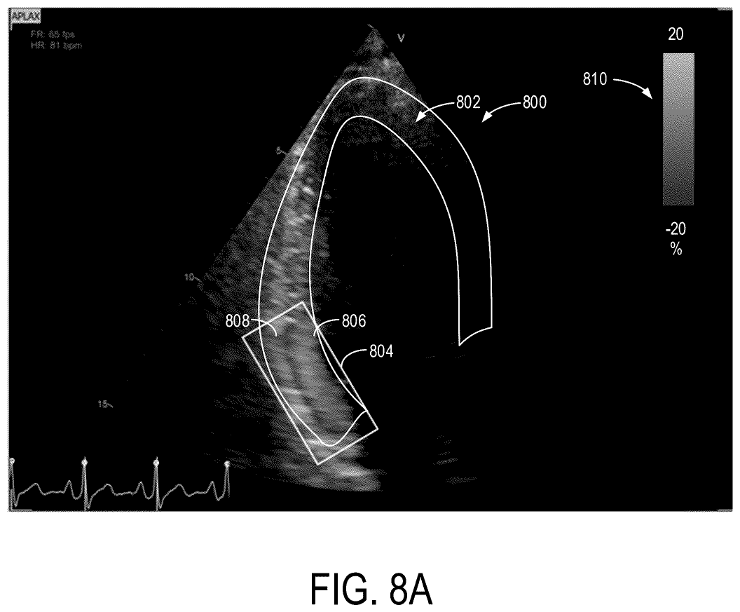

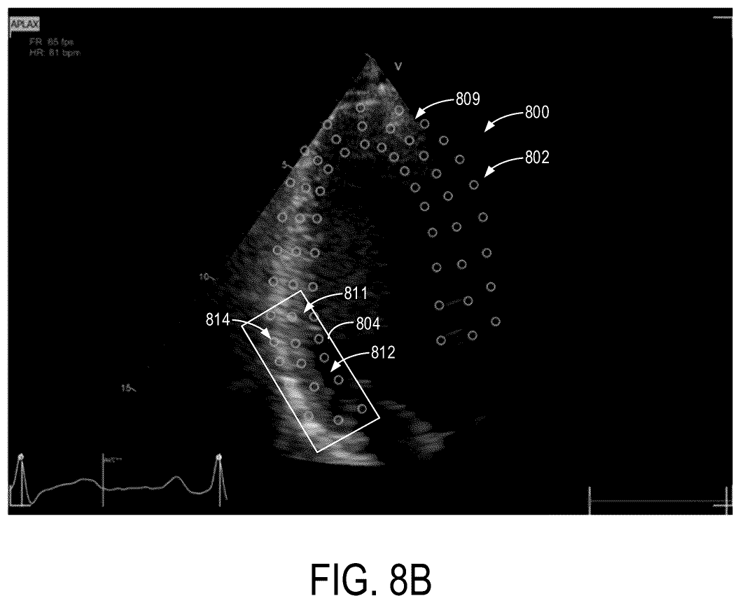

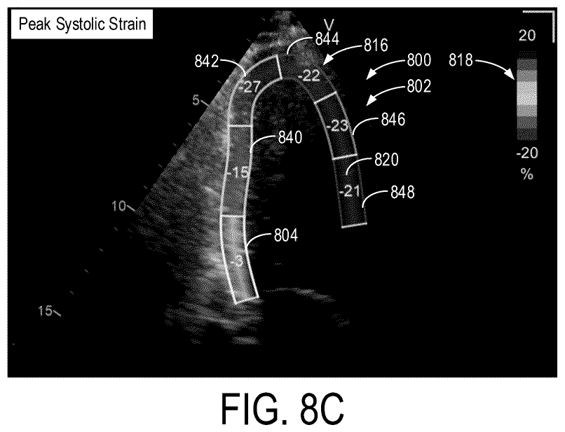

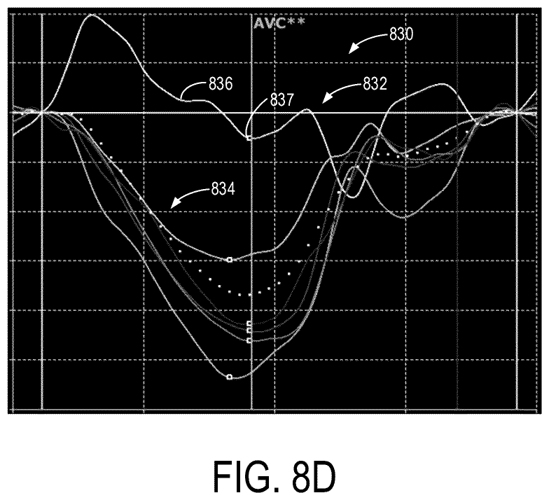

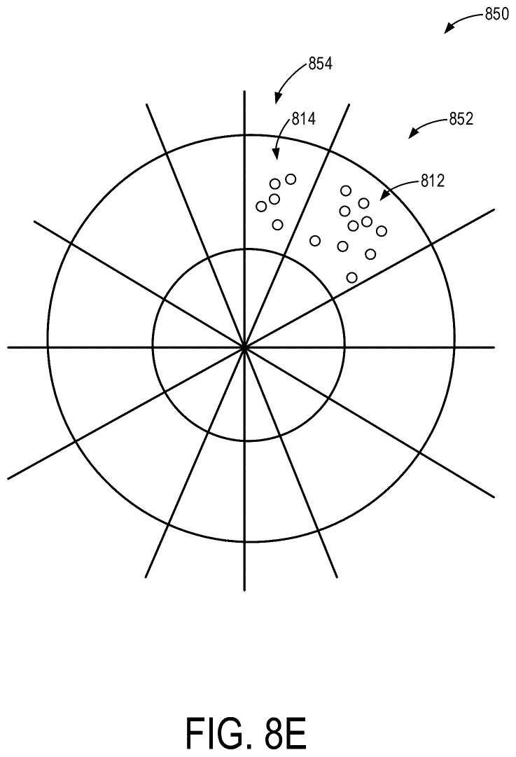

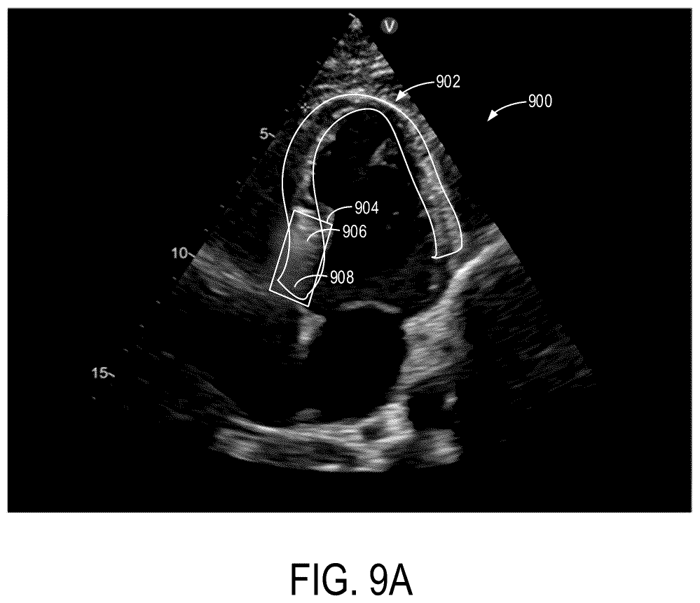

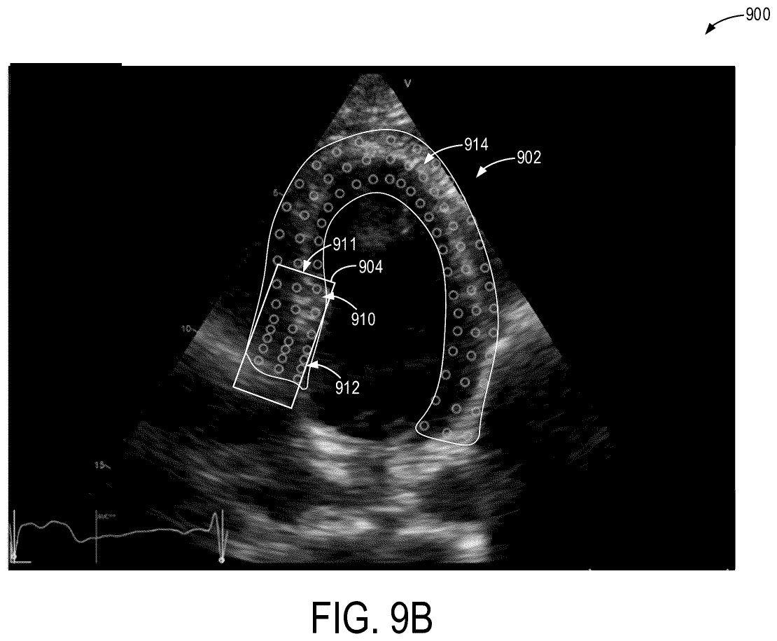

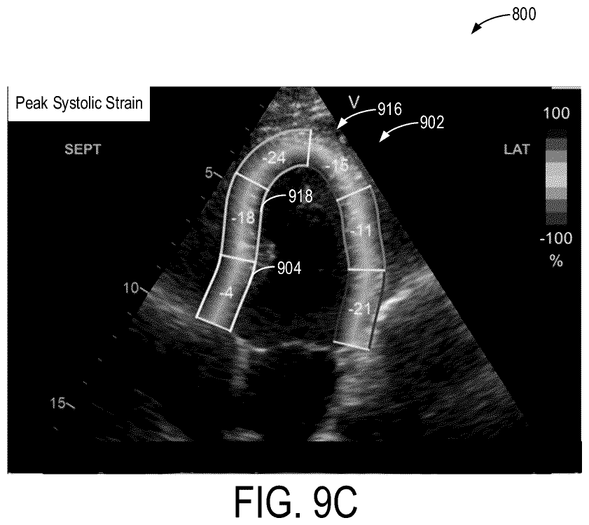

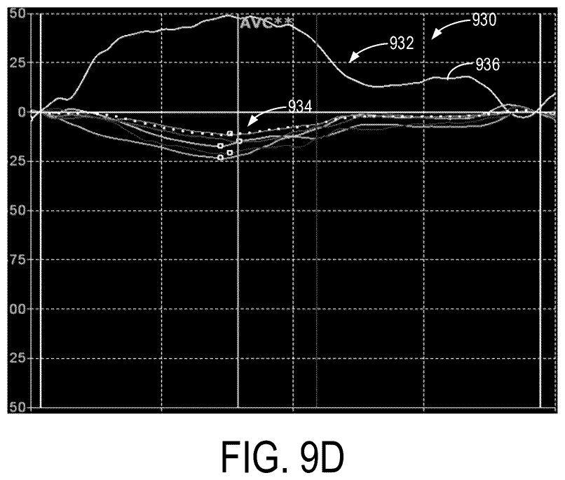

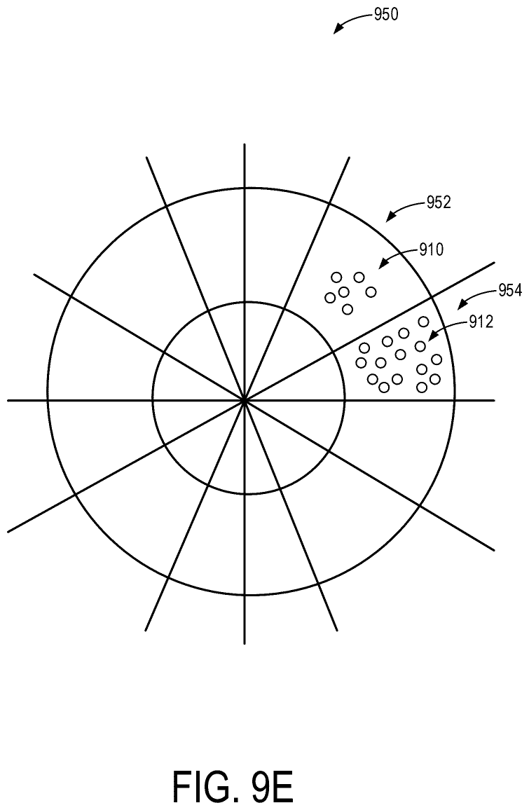

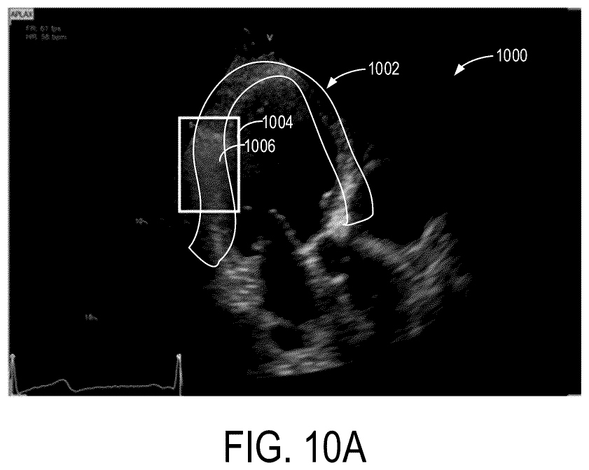

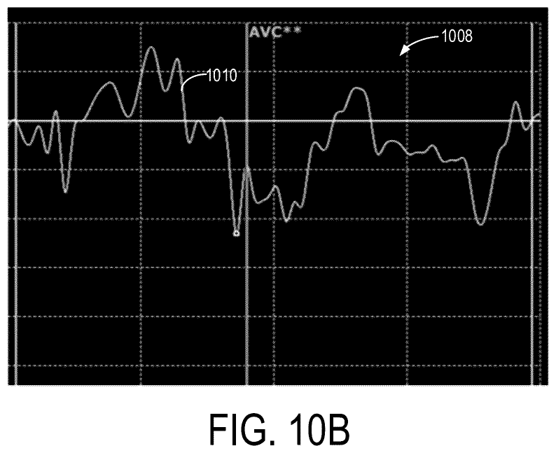

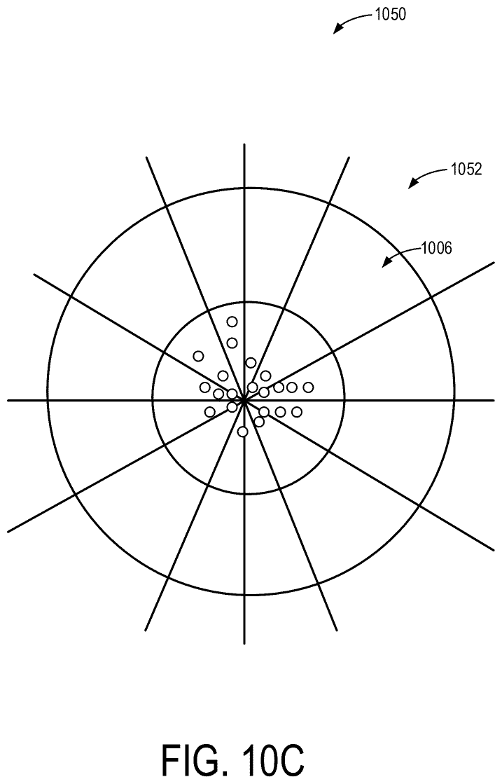

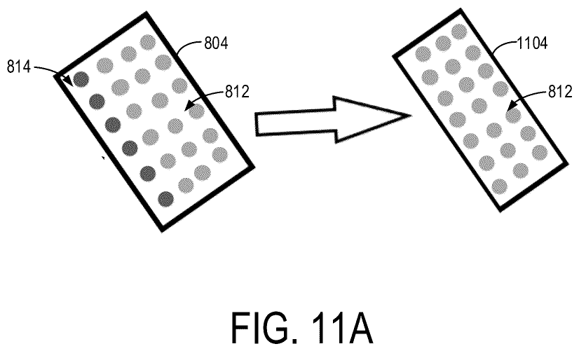

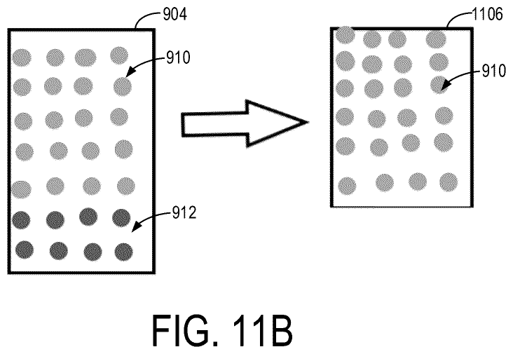

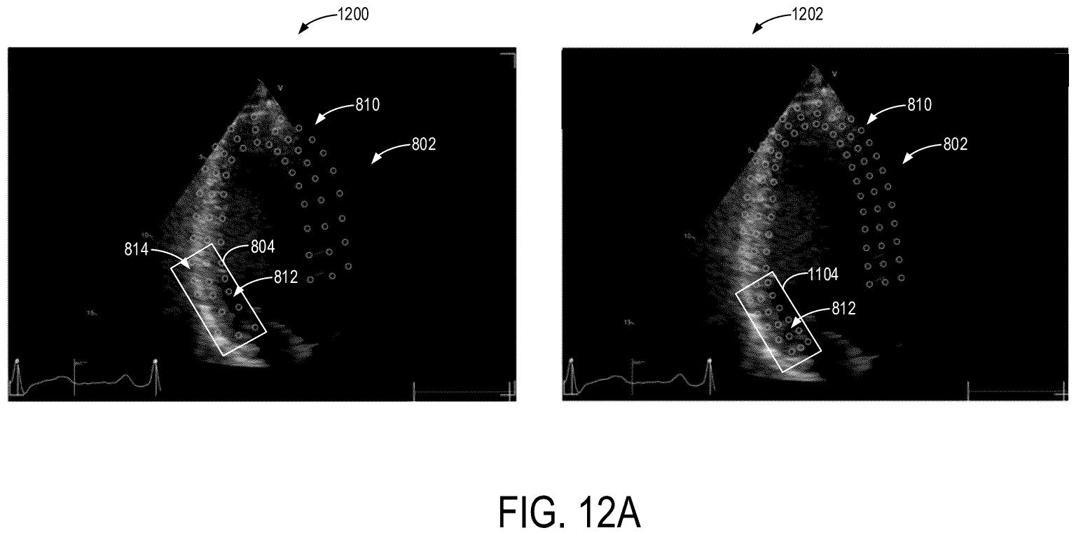

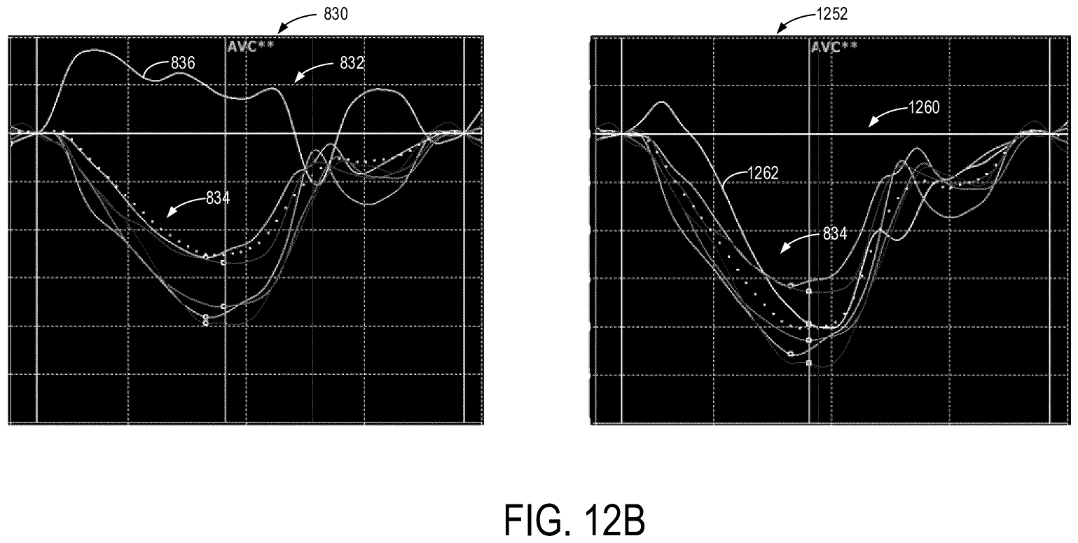

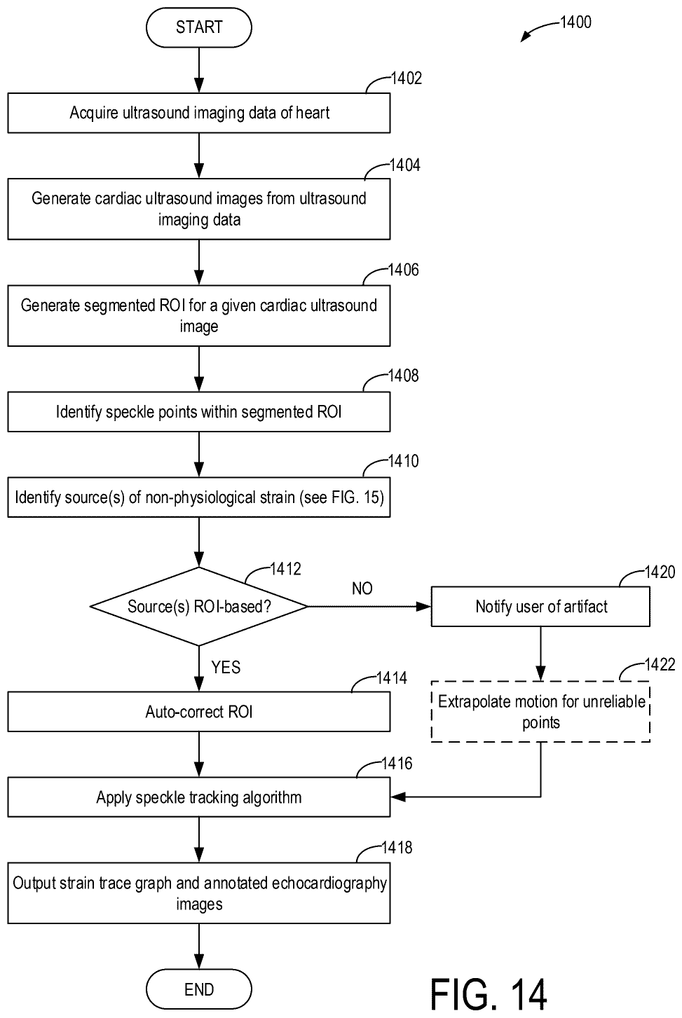

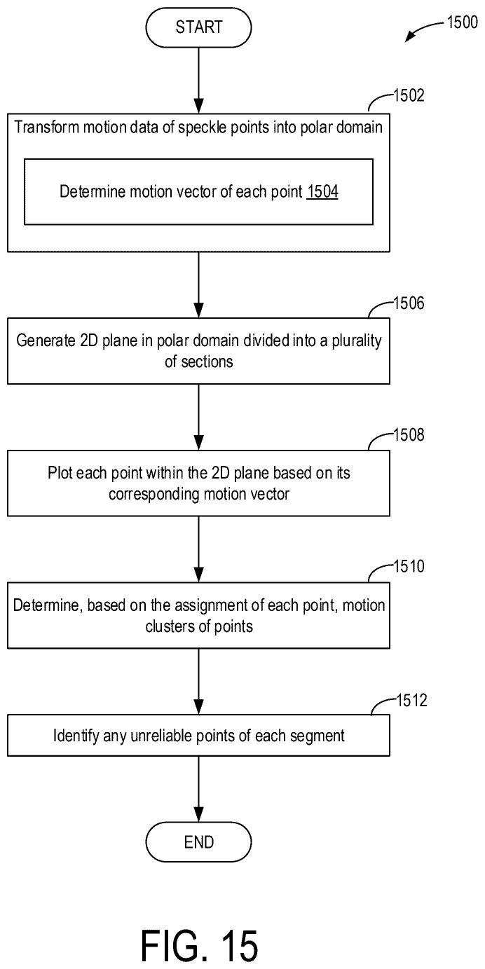

Systems and methods are herein provided that at least partially address the aforementioned issues by detecting non-physiologic strain traces and, when the source of the non-physiological strain traces is ROI-based, correcting the ROI positioning to correct the strain traces. Thus, according to embodiments described herein, images of the heart may be acquired by an ultrasound imaging system, such as the ultrasound imaging system shown in FIG. 1. An example image processing system that may be used to detect and correct non-physiological strain traces is shown in FIG. 2. A first example ultrasound image is shown in FIGS. 3 and 4 and example strain trace plots of the first example ultrasound image are shown in FIGS. 5 and 6. An ROI of an ultrasound image may be partitioned into predefined segments, wherein each segment includes a plurality of points (e.g., speckles) of the ROI. The points of each segment, when strain is physiological, may move with similar vectors, thus defining a motion cluster. However, the points in a segment that includes sources of non-physiological strain may include more than one motion cluster. An example segment with points in a single motion cluster and an example segment with points in more than one motion cluster are shown in FIG. 7A. Detection of non-physiological strain, as herein disclosed, is based on detection of motion vectors and motion clusters. Motion vectors of each point of each segment may be transformed into a two-dimensional (2D) plane of a polar domain (or 3D plane of a spherical domain, in the case of 3D strain calculations) to determine motion clustering. Segments with points with motion vectors that are clustered into one section of the 2D plane may be thus have physiological strain while segments with points in multiple sections of the 2D plane may have non-physiological strain. Example 2D planes for the segments of FIG. 7A are shown in FIG. 7B. Examples of ultrasound images with non-physiological strain trace due to ROI malpositioning, corresponding strain trace plots, and corresponding 2D planes in the polar domain are shown in FIGS. 8A-8E and 9A-9E. An example of an ultrasound image with non-physiological strain trace due to image artifact and a corresponding strain trace plot are shown in FIGS. 10A and 10B. A 2D plane for the motion vectors of points of the ultrasound image of FIG. 10A is shown in FIG. 10C. Examples of ROI-based correction are shown in FIGS. 11A-B. ROI-based correction for the examples shown in FIGS. 8A-E and 9A-E are shown in FIGS. 12A-B and 13A-B, respectively. A method for speckle tracking echocardiography with correction of non-physiological strain trace is shown in a flowchart in FIG. 14 and a method for identifying sources of non-physiological strain is shown in a flowchart in FIG. 15.

Advantages that may be realized in the practice of some embodiments of the described systems and techniques are that areas of healthy function and areas of impaired muscle function may be more easily identified. For example, more accurate strain value calculations may make it easier for a clinician to distinguish healthy regions that experience strong contraction, and thus more strain, from regions having impaired contraction, and thus less strain. In contrast, underestimating the strain values by including the pericardium may obscure the actual regions of impaired muscle function by showing larger areas of impaired function even in healthy tissue. Further identifying, and in some cases correcting for, artifact that would otherwise skew calculated strain, may increase accuracy of calculation and reduce need for repeat images, thereby saving user time and effort. Incorporating identification and correction of non-physiological strain sources into the process of strain calculation may thus reduce the need for repeated ROI segmentation, strain calculation, and repeated scan acquisition, thereby increasing the overall efficiency of the system. Further, the systems and techniques described herein may reduce variability between users and between exams, as any pericardium or artifact erroneously included in the region of interest by the user may not be included in the strain value calculations. Overall, more accurate and timely diagnoses may be obtained.

Although the systems and methods described below for evaluating medical images are discussed with reference to an ultrasound imaging system, it may be noted that the methods described herein may be applied to a plurality of imaging systems. As the processes described herein may be applied to pre-processed imaging data and/or to processed images, the term “image” is generally used throughout the disclosure to denote both pre-processed and partially-processed image data (e.g., pre-beamformed radio frequency or in-phase/quadrature data, pre-scan converted radio frequency data) as well as fully processed images (e.g., scan converted and filtered images ready for display).

Referring to FIG. 1, a schematic diagram of an ultrasound imaging system 100 in accordance with an embodiment of the disclosure is shown. However, it may be understood that embodiments set forth herein may be implemented using other types of medical imaging modalities (e.g., magnetic resonance imaging, computed tomography, positron emission tomography, and so on). The ultrasound imaging system 100 includes a transmit beamformer 101 and a transmitter 102 that drives elements (e.g., transducer elements) 104 within a transducer array, herein referred to as a probe 106, to emit pulsed ultrasonic signals (referred to herein as transmit pulses) into a body (not shown). According to an embodiment, the probe 106 may be a one-dimensional transducer array probe. However, in some embodiments, the probe 106 may be a two-dimensional matrix transducer array probe. The transducer elements 104 may be comprised of a piezoelectric material. When a voltage is applied to the piezoelectric material, the piezoelectric material physically expands and contracts, emitting an ultrasonic spherical wave. In this way, the transducer elements 104 may convert electronic transmit signals into acoustic transmit beams. While not specifically shown in FIG. 1, the probe 106 may comprise a matching layer configured to reduce acoustic impedance mismatch between a piezoelectric resonator and the subject, a backing configured to control sensitivity and bandwidth of the probe 106, and a damping block configured to absorb ultrasound energy and stray signals from a housing of the probe 106.

After the transducer elements 104 of the probe 106 emit pulsed ultrasonic signals into the body (of a patient), the pulsed ultrasonic signals are back-scattered from structures within an interior of the body, like blood cells and muscular tissue, to produce echoes that return to the elements 104. The echoes are converted into electrical signals, or ultrasound data, by the elements 104, and the electrical signals are received by a receiver 108. The electrical signals representing the received echoes are passed through a receive beamformer 110 that performs beamforming and outputs ultrasound data, which may be in the form of a radiofrequency (RF) signal. Additionally, the transducer elements 104 may produce one or more ultrasonic pulses to form one or more transmit beams in accordance with the received echoes.

According to some embodiments, the probe 106 may contain electronic circuitry to do all or part of the transmit beamforming and/or the receive beamforming. For example, all or part of the transmit beamformer 101, the transmitter 102, the receiver 108, and the receive beamformer 110 may be positioned within the probe 106. The terms “scan” or “scanning” may also be used in this disclosure to refer to acquiring data through the process of transmitting and receiving ultrasonic signals. The term “data” may be used in this disclosure to refer to one or more datasets acquired with an ultrasound imaging system. In one embodiment, data acquired via the ultrasound imaging system 100 may be processed via an imaging processing system, as will be elaborated below with respect to FIG. 2.

A user interface 115 may be used to control operation of the ultrasound imaging system 100, including to control the input of patient data (e.g., patient medical history), to change a scanning or display parameter, to initiate a probe repolarization sequence, and the like. The user interface 115 may include one or more of a rotary element, a mouse, a keyboard, a trackball, hard keys linked to specific actions, soft keys that may be configured to control different functions, and a graphical user interface displayed on a display device 118. In some embodiments, the display device 118 may include a touch-sensitive display, and thus, the display device 118 may be included in the user interface 115. In some embodiments, the user interface 115 may further include an audio system, such as one or more speakers, to output sound.

The ultrasound imaging system 100 also includes a processor 116 to control the transmit beamformer 101, the transmitter 102, the receiver 108, and the receive beamformer 110. The processor 116 is in electronic communication (e.g., communicatively connected) with the probe 106. As used herein, the term “electronic communication” may be defined to include both wired and wireless communications. The processor 116 may control the probe 106 to acquire data according to instructions stored on a memory of the processor and/or a memory 120. As one example, the processor 116 controls which of the elements 104 are active and the shape of a beam emitted from the probe 106. The processor 116 is also in electronic communication with the display device 118, and the processor 116 may process the data (e.g., ultrasound data) into images for display on the display device 118. The processor 116 may include a central processing unit (CPU), according to an embodiment. According to other embodiments, the processor 116 may include other electronic components capable of carrying out processing functions, such as a digital signal processor, a field-programmable gate array (FPGA), or a graphics board. According to other embodiments, the processor 116 may include multiple electronic components capable of carrying out processing functions. For example, the processor 116 may include two or more electronic components selected from a list of electronic components including: a central processor, a digital signal processor, a field-programmable gate array, and a graphics board. According to another embodiment, the processor 116 may also include a complex demodulator (not shown) that demodulates RF data and generates raw data. In another embodiment, the demodulation may be carried out earlier in the processing chain.

The processor 116 is adapted to perform one or more processing operations according to a plurality of selectable ultrasound modalities on the data. In one example, the data may be processed in real-time during a scanning session as the echo signals are received by receiver 108 and transmitted to processor 116. For the purposes of this disclosure, the term “real-time” is defined to include a procedure that is performed without any intentional delay (e.g., substantially at the time of occurrence). For example, an embodiment may acquire images at a real-time rate of 7-20 frames/sec. The ultrasound imaging system 100 may acquire 2D data of one or more planes at a significantly faster rate. However, it should be understood that the real-time frame-rate may be dependent on a length (e.g., duration) of time that it takes to acquire and/or process each frame of data for display. Accordingly, when acquiring a relatively large amount of data, the real-time frame-rate may be slower. Thus, some embodiments may have real-time frame-rates that are considerably faster than 20 frames/sec while other embodiments may have real-time frame-rates slower than 7 frames/sec.

In some embodiments, the data may be stored temporarily in a buffer (not shown) during a scanning session and processed in less than real-time in a live or off-line operation. Some embodiments of the disclosure may include multiple processors (not shown) to handle the processing tasks that are handled by the processor 116 according to the exemplary embodiment described hereinabove. For example, a first processor may be utilized to demodulate and decimate the RF signal while a second processor may be used to further process the data, for example by augmenting the data as described further herein, prior to displaying an image. It should be appreciated that other embodiments may use a different arrangement of processors.

The ultrasound imaging system 100 may continuously acquire data at a frame-rate of, for example, 10 Hertz (Hz) to 30 Hz (e.g., 10 to 30 frames per second). Images generated from the data may be refreshed at a similar frame-rate on the display device 118. Other embodiments may acquire and display data at different rates. For example, some embodiments may acquire data at a frame-rate of less than 10 Hz or greater than 30 Hz depending on the size of the frame and the intended application. The memory 120 may store processed frames of acquired data. In an exemplary embodiment, the memory 120 is of sufficient capacity to store at least several seconds' worth of frames of ultrasound data. The frames of data are stored in a manner to facilitate retrieval thereof according to its order or time of acquisition. The memory 120 may comprise any known data storage medium.

In various embodiments of the present disclosure, data may be processed in different mode-related modules by the processor 116 (e.g., B-mode, Color Doppler, M-mode, Color M-mode, spectral Doppler, elastography, tissue velocity imaging, strain, strain rate, and the like) to form 2D or three-dimensional (3D) images. When multiple images are obtained, the processor 116 may also be configured to stabilize or register the images. For example, one or more modules may generate B-mode, color Doppler, M-mode, color M-mode, color flow imaging, spectral Doppler, elastography, tissue velocity imaging (TVI), strain (e.g., speckle tracking echocardiography), strain rate, and the like, and combinations thereof. As one example, the one or more modules may process B-mode data, which may include 2D or 3D B-mode data, and the like. The image lines and/or frames are stored in memory and may include timing information indicating a time at which the image lines and/or frames were stored in memory. The modules may include, for example, a scan conversion module to perform scan conversion operations to convert the acquired images from beam space coordinates to display space coordinates. A video processor module may be provided that reads the acquired images from a memory and displays an image loop (e.g., cine loop) in real-time while a procedure (e.g., ultrasound imaging) is being performed on the patient. The video processor module may include a separate image memory, and the ultrasound images may be written to the image memory in order to be read and displayed by the display device 118.

Further, the components of the ultrasound imaging system 100 may be coupled to one another to form a single structure, may be separate but located within a common room, or may be remotely located with respect to one another. For example, one or more of the modules described herein may operate in a data server that has a distinct and remote location with respect to other components of the ultrasound imaging system 100, such as the probe 106 and the user interface 115. Optionally, the ultrasound imaging system 100 may be a unitary system that is capable of being moved (e.g., portably) from room to room. For example, the ultrasound imaging system 100 may include wheels or may be transported on a cart, or may comprise a handheld device.

For example, in various embodiments of the present disclosure, one or more components of the ultrasound imaging system 100 may be included in a portable, handheld ultrasound imaging device. For example, the display device 118 and the user interface 115 may be integrated into an exterior surface of the handheld ultrasound imaging device, which may further contain the processor 116 and the memory 120 therein. The probe 106 may comprise a handheld probe in electronic communication with the handheld ultrasound imaging device to collect raw ultrasound data. The transmit beamformer 101, the transmitter 102, the receiver 108, and the receive beamformer 110 may be included in the same or different portions of the ultrasound imaging system 100. For example, the transmit beamformer 101, the transmitter 102, the receiver 108, and the receive beamformer 110 may be included in the handheld ultrasound imaging device, the probe, and combinations thereof.

Referring now to FIG. 2, an example medical image processing system 200 is shown. In some embodiments, the medical image processing system 200 is incorporated into a medical imaging system, such as an ultrasound imaging system (e.g., the ultrasound imaging system 100 of FIG. 1), a magnetic resonance imaging (MRI) system, a computed tomography (CT) system, a single-photon emission computed tomography (SPECT) system, and the like. In some embodiments, at least a portion of the medical image processing system 200 is disposed at a device (e.g., an edge device or server) communicably coupled to the medical imaging system via wired and/or wireless connections. In some embodiments, the medical image processing system 200 is disposed at a separate device (e.g., a workstation) that can receive images from the medical imaging system or from a storage device that stores the images generated by the medical imaging system. The medical image processing system 200 may comprise an image processor 231, a user input device 232, and a display device 233. For example, the image processor 231 may be operatively/communicatively coupled to the user input device 232 and the display device 233.

The image processor 231 includes a processor 204 configured to execute machine readable instructions stored in a non-transitory memory 206. The processor 204 may be single core or multi-core, and the programs executed by the processor 204 may be configured for parallel or distributed processing. In some embodiments, the processor 204 may optionally include individual components that are distributed throughout two or more devices, which may be remotely located and/or configured for coordinated processing. In some embodiments, one or more aspects of the processor 204 may be virtualized and executed by remotely-accessible networked computing devices configured in a cloud computing configuration. In some embodiments, the processor 204 may include other electronic components capable of carrying out processing functions, such as a digital signal processor, a FPGA, or a graphics board. In some embodiments, the processor 204 may include multiple electronic components capable of carrying out processing functions. For example, the processor 204 may include two or more electronic components selected from a plurality of possible electronic components, including a central processor, a digital signal processor, a field-programmable gate array, and a graphics board. In still further embodiments, the processor 204 may be configured as a graphical processing unit (GPU), including parallel computing architecture and parallel processing capabilities.

In the embodiment shown in FIG. 2, the non-transitory memory 206 stores a strain trace analyzer 212 and medical image data 214. The strain trace analyzer 212 includes one or more algorithms configured to process input medical images from the medical image data 214. Specifically, the strain trace analyzer 212 may ingest medical image data, including motion data of points of an ROI, to define motion clusters. Based on the motion clusters, the strain trace analyzer 212 may determine physiological strain trace vs non-physiological strain trace for each segment of the ROI. As an example, the strain trace analyzer 212 may include instructions for transforming motion vectors of individual points of each segment to a 2D plane in the polar domain (or 3D plane in the spherical domain in 3D applications) in order to define motion clusters. It should be understood that while 2D planes in the polar domain are largely herein described, 3D planes in the spherical domain may also be utilized in 3D imaging applications.

Further, the strain trace analyzer 212 may include predefined parameters of 2D planes, for example, 2D planes may be partitioned into a plurality of predefined sections. The strain trace analyzer 212 may use the predefined sections in order to plot motion vectors of points of each segment. The strain trace analyzer 212 may then define motion clusters as a subset of points that reside within a particular section of the 2D plane. The strain trace analyzer 212 may analyze the motion clusters to determine whether subsets of points of a segment of the ROI are outliers with respect to a given motion cluster to which the points of the segment correspond, as will be further described below. Outlying motion vectors may be identified as unreliable motion vectors, and the speckle points to which those motion vectors correspond may thus be unreliable points. In this way, when outlying motion vectors for a segment are identified, the segment may be determined to have non-physiological strain trace. When outlying motion vectors are not identified in any segment of the ROI, the ROI may be determined to have physiological strain trace.

Further still, the strain trace analyzer 212 may comprise instructions for determining whether identified non-physiological strain trace is ROI-based or artifact-based. As an example, two motion clusters may be identified for a segment, ROI-based non-physiological strain may be identified. When various motion clusters or scattered motion vectors are identified for a segment, the non-physiological strain trace may be artifact-based. Further, when motion vectors are centered about the origin of the 2D plane, the non-physiological strain trace may be artifact-based. When the source of non-physiological strain is identified as ROI-based, an ROI correction module 216 may correct the ROI to remove the non-physiological strain trace by removing the unreliable speckle points from the ROI, as will be further explained below. In some examples, removal of non-physiological strain traces may include replacement of non-physiological strain traces with physiological strain traces, when ROI-based correction is possible. As an example, the ROI correction module 216 may remove the outlying points of the segment so that only the points within the corresponding motion cluster are used for determining strain trace thereof. In some embodiments, the strain trace analyzer 212 may evaluate the medical image data 214 as it is acquired in real-time. In some examples, when the source of non-physiological strain is identified as artifact-based, the ROI correction module 216 may employ extrapolation algorithms to extrapolate motion for the corresponding speckle points from neighboring speckle points. Additionally or alternatively, the strain trace analyzer 212 may evaluate the medical image data 214 offline, not in real-time.

In some embodiments, the strain trace analyzer 212 may further include trained and/or untrained neural networks for identifying and segmenting myocardium (e.g., cardiac muscle) in the medical image data 214 in addition to trained and/or untrained neural networks for identify and segmenting the pericardium. Additionally or alternatively, separate cardiac segmentation modules may be included within, or may be accessed by, the image processor 231. For example, the image processor 231 may use a cardiac segmentation module in order to generate or otherwise determine the ROI.

The non-transitory memory 206 further stores the medical image data 214. The medical image data 214 may include, for example, functional and/or anatomical images captured by an imaging modality, such as ultrasound imaging systems, MRI systems, CT systems, and so forth. As one example, the medical image data 214 may include ultrasound images, such as cardiac ultrasound images. Further, the medical image data 214 may include one or more of 2D images, 3D images, static single frame images, and multi-frame cine-loops (e.g., movies).

In some embodiments, the non-transitory memory 206 may include components disposed at two or more devices, which may be remotely located and/or configured for coordinated processing. In some embodiments, one or more aspects of the non-transitory memory 206 may include remotely-accessible networked storage devices in a cloud computing configuration. As one example, the non-transitory memory 206 may be part of a picture archiving and communication system (PACS) that is configured to store patient medical histories (e.g., electronic medical records), imaging data, test results, diagnosis information, management information, and/or scheduling information, for example.

The medical image processing system 200 may further include the user input device 232. The user input device 232 may comprise one or more of a touchscreen, a keyboard, a mouse, a trackpad, a motion sensing camera, or other device configured to enable a user to interact with and manipulate data stored within the image processor 231. As an example, the user input device 232 may enable a user to select images for analysis by the strain trace analyzer 212.

The display device 233 may include one or more display devices utilizing any type of display technology. In some embodiments, the display device 233 may comprise a computer monitor and may display unprocessed images, processed images, parametric maps, and/or exam reports. The display device 233 may be combined with the processor 204, the non-transitory memory 206, and/or the user input device 232 in a shared enclosure or may be a peripheral display device. The display device 233 may include a monitor, a touchscreen, a projector, or another type of display device, which may enable a user to view medical images and/or interact with various data stored in the non-transitory memory 206. In some embodiments, the display device 233 may be included in a mobile device, such as a smartphone, a tablet, a smartwatch, or the like.

It may be understood that the medical image processing system 200 shown in FIG. 2 is one non-limiting embodiment of an image processing system, and other imaging processing systems may include more, fewer, or different components without departing from the scope of this disclosure. Further, in some embodiments, at least portions of the medical image processing system 200 may be included in the ultrasound imaging system 100 of FIG. 1, or vice versa (e.g., at least portions of the ultrasound imaging system 100 may be included in the medical image processing system 200). Additionally, the processes and methods referred to herein may also be applied to other imaging systems, such as contrast echocardiography imaging systems, MRI, and the like.

Turning now to FIG. 3, a first example display output 300 is shown. The first example display output 300 may be displayed on a display device (e.g., display device 118) of an ultrasound imaging system (e.g., ultrasound imaging system 100). In other examples, the first display output 300 may be displayed on a display device of a care provider device (e.g., a desktop computer, a laptop computer, a tablet, a smart phone, etc.) configured to display medical images stored in memory (e.g., accessed via PACS). The display output 300 may include a cardiac ultrasound image 302. The cardiac ultrasound image 302 as shown may be a B-mode image. The display output 300 may include an image view 312 that indicates the view that is currently displayed. For example, the cardiac ultrasound image 302 may be an apical long axis (APLAX) view of the heart. The cardiac ultrasound image 302 may correspond to a patient. In some examples, the cardiac ultrasound image 302 may be displayed on the display device within the display output 300 in real time as the image is acquired. In other examples, the cardiac ultrasound image 302 may be displayed at a later time, for example when a radiologist or other care provider views the image for evaluation purposes.

The display output 300 may include an ROI 304. The ROI 304 may comprise a plurality of points 306, which may be identified as part of a speckle tracking echocardiography protocol. Points, also referred to herein as speckle points, may be brighter pixel “speckles” that are identified, for example by a speckle tracking algorithm. In some examples, the ROI 304 may be user defined. For example, a user may drag a cursor over the cardiac ultrasound image 302 to identify the ROI 304. In other examples, the ROI 304 may be defined by a segmentation model. In yet further examples, the ROI 304 may be defined based on a combination of both user inputs and a segmentation model. The ROI ideally corresponds to contractile myocardium tissue of the heart, however in some examples, pericardium, valvular structures, blood pool, and the like may be erroneously included in the ROI. Myocardium may result in physiological strain during contraction and relaxation, while pericardium and other structures or interferences may result in non-physiological strain. The strain values that are determined via the speckle tracking echocardiography may be indicated in an outputted annotated image, as is shown in FIG. 3, within the ROI 304 via a shading 308. In some examples, different shades or colors of the shading 308 may indicate different strain values, as will be further described below.

The display output 300 may further include a rhythm 310. The rhythm 310 may be a readout of the patient's cardiac rhythm as the cardiac ultrasound image 302 is acquired. In some examples, the cardiac ultrasound image 302 may be acquired over a period of time that includes one or more cardiac cycles. Each cardiac cycle comprises one heartbeat, which includes one period of diastole and one period of systole. During diastole, the myocardium relaxes and the heart refills with blood. During systole, the myocardium contracts to pump the blood out of the heart. The contraction and relaxation cause shortening and elongation of the myocardium, respectively, which may be quantified via the speckle tracking echocardiography. In some examples, the cardiac ultrasound image 302 may be a motive image (e.g., including a plurality of frames) that changes over the course of the imaged cardiac cycle(s). The rhythm 310 may indicate where in the cardiac cycle the currently displayed frame of the image is via a marker.

Turning now to FIG. 4, a second display output 400 is shown. The second display output 400 may display the same cardiac ultrasound image 302 as the first display output 300. The second display output 400 may include a segmented ROI overlay 404. The segmented ROI overlay 404 may indicate a plurality of segments 406 of the ROI (e.g., ROI 304). For example, the ROI may include a first segment 408, a second segment 410, and a third segment 412. Each of the segments may comprise a subset of the plurality of points 306.

Based on the speckle tracking echocardiography, strain may be determined for each of the segments. As such, in the annotated cardiac ultrasound image output, the shading 308 displayed within each segment of the ROI may correspond to the strain determined for the corresponding segment. In some examples, as is shown in FIG. 4, the strain value may be numerically indicated visually within each segment. The color or shade of the shading 308 displayed within each of the plurality of segments 406 may correspond to a key 414. The key 414 may comprise a gradient of colors or shades that each correspond to a strain value (e.g., in percentages).

Turning now to FIG. 5, a first strain trace graph 500 corresponding to the cardiac ultrasound image 302 is shown. In some examples, the first strain trace graph 500 may be displayed on the display device within the same user interface as the first and second display outputs 300, 400.

The first strain trace graph 500 may comprise a plurality of plots 504. Each of the plurality of plots 504 may correspond to one of the segments of the plurality of segments 406. Each of the plurality of plots 504 may plot strain over time over the course of the cardiac cycle that is imaged in the cardiac ultrasound image 302. For example, a first plot 508 may correspond to the first segment 408 and a second plot 510 may correspond to the second segment 410. The first strain trace graph 500 may thus indicate strain of different areas of the myocardium of the heart throughout the cardiac cycle.

A y-axis 502 of the first strain trace graph 500 may indicate level of strain (e.g., in percentage). An x-axis 503 may indicate position within the cardiac cycle. In some examples, an atrioventricular contraction (AVC) point 506 may be specifically labeled. The AVC point 506 may generally correspond to the peak systolic strain, thus the peak systolic strain may be correspond to the point at which each plot crosses the AVC point 506. In the plot shown in FIG. 5, the peak systolic strain is near the lowest point, wherein each of the plurality of plots 504 crosses the AVC point 506 near its nadir.

The strain traces plotted in the first strain trace graph 500 and other strain trace graphs herein described may indicate both cardiac morphology of the patient as well as whether any of the segments include non-physiological strain. For example, the first strain trace graph 500 may be an example of strain traces that are generated in a healthy patient with a typical cardiac cycle and contraction/relaxation cycle. The plurality of plots 504 may follow generally a similar pattern with the changes in strain over time, indicating that the strain determined by the speckle tracking echocardiography is physiologic.

In comparison, a second strain trace graph 600 is shown in FIG. 6 that corresponds to a patient with cardiac pathology. Similar to the first strain trace graph 500, the second strain trace graph 600 may include a y-axis 602 indicating strain (e.g., in percentage) increasing from bottom to top and an x-axis 603 indicating time over the course of a cardiac cycle. The second strain trace graph 600 may include a plurality of plots 604, each corresponding to a segment of an ROI of a cardiac ultrasound image. For example, when the second strain trace graph 600 corresponds to the cardiac ultrasound image 302, each of the plurality of plots 604 may correspond to one of the plurality of segments 406. For example, a first plot 608 may correspond to the first segment 408 and a second plot 610 may correspond to the second segment 410.

Strain, in patients with cardiac pathology such as congestive heart failure (CHF) in which the myocardium does not contract normally, may have less variation over the course of the cardiac cycle compared to a healthy patient with normal contraction and relaxation. Accordingly, the second strain trace graph 600 may indicate flatter plots than the plots of the first strain trace graph 500. In the event of inclusion of non-physiological strain, the resulting outputted strain trace graph may be unreliable. Thus, while a plot of the plurality of plots may appear flatter, as would usually suggest some type of cardiac pathology, the flatter appearance may be a result of the non-physiological strain rather than true pathology. In this way, non-physiological strain may affect diagnostic use of speckle tracking echocardiography and correction of non-physiological strain, as is herein disclosed, may increase the accuracy of speckle tracking echocardiography.

Turning now to FIGS. 7A and 7B, an example annotated cardiac ultrasound image and corresponding 2D plane transformation of points of a segment of an ROI of the cardiac ultrasound image are shown. In particular, FIG. 7A shows a third display output 700. Similar to the first and second display outputs described above, the third display output 700 may be displayed on a display device. For example, the third display output 700 may be displayed on a display device of an ultrasound system (e.g., display device 118) in real-time with acquisition of a cardiac image or on a display device of a care provider device (e.g., a laptop computer, a desktop computer, or the like).

The third display output 700 may comprise a cardiac ultrasound image 702. The cardiac ultrasound image may be annotated by a combination of user inputs and segmentation algorithms, in some examples. For example, an ROI may be designated. As described previously, the ROI may be partitioned into a plurality of segments each comprising a plurality of points identified via speckle tracking echocardiography. For example, a first segment 704 may comprise a first plurality of points 706 and a second segment 708 may comprise a second plurality of points 710.

As described above, each of the points of each segment may have a motion vector. In physiological strain, the motion vector of each point in a given segment may be within a range of length and angle and thus may share a given motion profile. In non-physiological strain, the motion vector of one or more points in a given segment may be outside a given range and thus the outlying points may not share a given profile of other points of the segment. For example, the first plurality of points of the first segment 704 may each comprise a motion vector that resides within a first specified range of length and angle. The second plurality of points 710, in contrast, may comprise one or more subsets of points with motion vectors residing within different ranges. For example, a first subset of points 712 may each have a motion vector within a second range and a second subset of points 714 may each have a motion vector within a third range. The different clusters of motion vectors may indicate that one or more of the points of the segment may generate non-physiological strain.

FIG. 7B shows 2D planes demonstrating transformation of the motion vectors of each of the points of the first and second segments 704, 708 into the polar domain. Based on length and angle of its motion vector, each point may be plotted within a respective 2D plane. For example, a first 2D plane 750 plots motion vectors of the first segment 704 and a second 2D plane 770 plots motion vectors of the second segment 708.

Each of the first and second 2D planes 750, 770 may be partitioned into a plurality of sections 752. The parameters (e.g., number of sections, position of sections, range of polar coordinates included in each section, etc.) of each of the plurality of sections 752 may be predefined in the system. In some examples, the parameters of the plurality of sections 752 may be adjustable based on user preferences, type of scan, patient demographics, patient history, or the like.

As an example, each of the first plurality of points 706 may have a motion vector that lies within a first section 754 of the first 2D plane 750. Thus, the first plurality of points 706 may comprise a first motion cluster. The second plurality of points 710 may comprise points with motion vectors that reside within multiple sections of the second 2D plane 770. For example, each of the first subset of points 712 may have a motion vector that lies within a second section 756 of the second 2D plane 770 and the second subset of points 714 may have a motion vector that lies within a third section 758 of the second 2D plane 770. Thus, the first subset of points 712 of the second plurality of points 710 may comprise a second motion cluster while the second subset of points 714 may comprise a third motion cluster. The second plurality of points 710 may thus comprise a plurality of motion clusters therein. When a section of an ROI comprises a plurality of motion clusters, non-physiological strain may be identified, as will be described with respect to FIGS. 8A-8E and 9A-9E below.

In this way, motion vectors for points of an ROI may be transformed into the polar domain in order to determine motion clusters of segments of the ROI. The identified motion clusters and motion profiles therein may indicate presence, and in some examples type of source of, non-physiological strain.

Turning now to FIGS. 8A-8E, an example cardiac ultrasound image including ROI-based non-physiological strain is shown. FIG. 8A shows a cardiac ultrasound image 800. FIG. 8B shows points of a segmented ROI 802 of the cardiac ultrasound image 800. FIGS. 8C-D show strain trace data of the cardiac ultrasound image 800, with FIG. 8C specifically showing an annotated cardiac ultrasound image including visual and textual representations of peak systolic strain of each segment of the segmented ROI 802 and FIG. 8D specifically showing a strain trace graph. FIG. 8E shows a 2D plane plotting motion vectors of points of the segmented ROI 802.

Starting with FIG. 8A, the cardiac ultrasound image 800 is shown. The cardiac ultrasound image 800 may be part of a display output displayed on a display device, such as a display device of an ultrasound system used to acquire the cardiac ultrasound image (e.g., display device 118 of FIG. 1) or a display device configured to display the image acquired by the ultrasound system, such as a care provider device configured to access PACS.

The cardiac ultrasound image 800 may be an annotated image, in some examples. For example, the cardiac ultrasound image 800 may include the segmented ROI 802. The segmented ROI 802 may be displayed as an overlay on the cardiac ultrasound image 800 within the display output. As previously noted, the segmented ROI 802 may comprise a plurality of segments, including first segment 804. The plurality of segments may be predefined, for example the plurality of segments may be of a known number, size, and may comprise a known amount of points within each segment. The motion of each point may be considered during speckle tracking echocardiography when determining strain as the cardiac muscle contracts and expands.

The peak systolic strain that is determined based on speckle tracking echocardiography applied to the cardiac ultrasound image 800 may also be displayed visually as an overlay on the cardiac ultrasound image 800. For example, the first segment 804 comprises a first strain 806 and a second strain 808. The first strain 806 and the second strain 808, determined via speckle tracking echocardiography, may have different values. For example, the first strain 806, as indicated via a key 810, may have a strain of closer to positive 20% while the second strain 808 may have a strain of closer to negative 20%. The shades indicated by the key 810 may be represented within the segmented ROI 802, similar to the shading 308 described above. The different shades within the same segment, e.g., the first segment 804, may indicate more than one motion cluster within the segment, thereby indicating sources of non-physiological strain within the segment.

FIG. 8B shows a plurality of points 809 within the segmented ROI 802. Each segment within the segmented ROI 802 may comprise a subset of the plurality of points 809. For example, the first segment 804 may comprise therein a first subset 811 of the plurality of points. Each of the plurality of points 809 is represented as a circle overlaid on the cardiac ultrasound image 800. Each of the points may indicate a positioned within the ROI that is tracked with the speckle tracking algorithm in order to determine strain. The points, as the heart contracts and expands, may move within the cardiac ultrasound image 800. Thus, each point may have a motion vector that is determined by the speckle tracking algorithm. Each motion vector may have a magnitude, length, and direction that indicates how the point moves. The difference in position of each point between systole and diastole (e.g., between contraction and relaxation) is strain, as previously discussed. Different motion profiles of points thus indicate different strain values, as a point that moves less far or in a different direction than another may result in a different amount of strain than the other.

As noted, the first segment 804 of the segmented ROI 802 has two different areas of strain, the first strain 806 and the second strain 808. The first segment 804 comprises a first subset 812 of the first subset of points 811 that corresponds to the first strain 806 and a second subset 814 of the first subset of points 811 that corresponds to the second strain 808. The first subset 812 of points may correspond to myocardium, or an area of the segmented ROI that corresponds to myocardium. The second subset 814 of points, however, may not correspond to myocardium. Rather, as an example, the second subset 814 may correspond to pericardium. Pericardium, by nature of not being a contractile tissue, may have different motion than the myocardium. While the pericardium may move as the heart contracts and expands, its motion vectors may different from the nearby myocardium. Thus, the strain calculated for the first and second subsets 812, 814 may differ, as is noted by the first strain 806 and the second strain 808. In some examples, pericardium may be included in the segmented ROI 802 due to inaccuracies in the applied segmentation algorithm, human error, and/or the like.

While the first and second strains 806, 808 are noted in FIG. 8A, the output of the speckle tracking algorithm may consider them together for the segment. With the different strains that are present within the first segment 804, the first segment 804 may have an overall strain outputted. Because of the contribution of strain calculated for the pericardium portion corresponding to the second subset 814 of points, the overall strain outputted may statistically differ from the strain calculated for the other segments, as is shown in FIG. 8C, and thus be inaccurate.

FIG. 8C shows visual and textual representations of the calculated peak systolic strain of each of the plurality of segments 816 of the segmented ROI 802 in an annotated cardiac ultrasound image. Shading 820 is displayed within the segmented ROI 802. As explained previously, the shading 802, per a key 818, visually indicates the amount of strain (peak systolic strain) within each segment.

For example, the amount of peak systolic strain, indicated both textually within the segments and by the shading 820, for second, third, fourth, fifth, and sixth segments 840, 842, 844, 846, and 848 is between −15% and −27% while the strain of the first segment 804 is −3%. As shown in FIGS. 8A and 8B, the first segment 804 comprises points with two motion clusters, resulting in the first strain 806 and the second strain 808. The second strain 808, as demonstrated by the shading in FIG. 8A, may be a positive strain of approximately +20%. Thus, the cumulative strain of the first segment 804 may be larger than the other segments of the segmented ROI 802.

A strain trace graph 830 is shown in FIG. 8D. The strain trace graph 830 comprises plurality of plots 832. Each of the plurality of plots 832 corresponds to one of the plurality of segments 816 of the cardiac ultrasound image 800. The strain trace graph 830 includes a y-axis that indicates percent strain and an x-axis that indicates time over the course of a cardiac cycle. Strain within each of the plurality of segments 816 is plotted as percent strain over time. The peak systolic strain is depicted as an indicator along each plot. For example, the peak systolic strain of the first segment 804 is depicted as indicator 837 along a first plot 836, wherein the first plot 836 corresponds to the first segment 804.

In the particular example shown, the plurality of plots 832 may include the first plot 836 and one or more second plots 834. The one or more second plots 834 may correspond to the second, third, fourth, fifth, and sixth segments 840, 842, 844, 846, and 848 as shown in FIG. 8C. As such, the peak systolic strain, designated along the plot with an indicator, may correspond to the displayed strains in FIG. 8C.

The plots of the one or more second plots 834 may generally follow a similar path of change in strain over time. The first plot 836, however, may not follow the similar path of the one or more second plots 834. This may be a result of the inclusion of the pericardium in the segmented ROI 802 in the first segment 804, which is a source of non-physiological strain. The non-physiological strain, by nature of resulting from portions of the heart with a different motion profile may thus affect the determined strain for the first segment 804. As herein described, the system disclosed may detect non-physiological strain, such as that produced by the second subset 814 due to the motion profile of the second subset 814.

While the strain trace graph 830, and other strain trace graphs herein described below, may distinctly show an outlying plot indicating non-physiological strain, in practicality, the outlying plot may be more subtle. Non-physiological strain traces may be difficult to ascertain in strain trace graph outputs with the human eye. Even if a user can discern a potential area of non-physiological strain, they cannot correct for it other than to repeat the scan and/or repeat segmentation of the ROI and calculation of strain via speckle tracking, which may increase time spent by the user as well as increase processing demands for the system with repeated acquisitions and/or segmentations.

FIG. 8E demonstrates, via a 2D plane 850 in the polar domain, the motion profiles of the first and second subsets 812, 814, as an example. Each of the first subset 812 of points of the first segment 804 may have a motion vector that lies within a first section 852 of the 2D plane 850 due to the motion vectors thereof having a similar motion profile. The first subset 812 of points may thus form a first motion cluster. Each of the second subset 814 of points may have a motion vector that lies within a second section 854 of the 2D plane 850. For example, the second subset 814 of points may correspond to areas of pericardium while the first subset 812 of points may correspond to areas of myocardium. The pericardium may have a different motion profile than the myocardium and thus the points corresponding to pericardium may have different motion vectors than points corresponding to myocardium. The second subset of points 814 may thus differ from the motion profile of the first subset 812 and may form a second motion cluster. The myocardium may produce physiological strain during contraction-elongation, however the pericardium my produce non-physiological strain. Thus, the system may detect non-physiological strain in instances in which multiple motion clusters are detected for a segment of the ROI. Correcting the ROI, in examples such as the example of FIGS. 8A-8E wherein the source of the non-physiological strain is due to ROI errors, may remove the non-physiological strain, as will be explained further below.

FIGS. 9A-9E present a similar example of ROI-based non-physiological strain. In particular, FIGS. 9A-9E show a second example cardiac ultrasound image with a segmented ROI and strain trace data thereof. First, FIG. 9A shows a cardiac ultrasound image 900. Second, FIG. 9B shows points of a segmented ROI 902 of the cardiac ultrasound image 900. Third, FIGS. 9C-D shows strain trace data of the cardiac ultrasound image 900, with FIG. 9C specifically showing visual and textual representations of peak systolic strain of each segment of the segmented ROI 902 and FIG. 8D specifically showing a strain trace graph. Fifth, FIG. 9E shows a 2D plane plotting motion vectors of points of the segmented ROI 902.

Similar to as described with respect to FIGS. 8A-8E, a first segment 904 of the segmented ROI 902 may comprise a first strain 906 and a second strain 908. At peak systolic strain, the first and second strains 906, 908 may be different values, indicating different motion profiles therebetween.

As shown in FIG. 9B, the segmented ROI 902 may comprise a plurality of points 914 that may be tracked by the speckle tracking algorithm. Each segment of the segmented ROI 902 may comprise a subset of the plurality of points. For example, the first segment 904 may comprise a first subset 911 of the plurality of points 914. A first subset 910 of the first subset 911 of the plurality of points 914 may correspond to the first strain 906. A second subset 912 of the first subset 911 of the plurality of points 914 may correspond to the second strain 908. The second subset 912 may have a different motion profile than the first subset 910.

For example, the second subset 912 may correspond to non-myocardium structure, such as blood pool or valve, similar to the area of pericardium included in the ROI of the cardiac ultrasound image 800 described above. The first subset 910, conversely, may correspond to myocardium. The myocardium, as contractile tissue of the heart, may be a source of physiological strain while the non-myocardium structure may be a source of non-physiological strain. The different motion profiles of the first and second subsets 910, 912 are indicated visually in FIG. 9B by the position of the points. The points of the second subset 912 are positioned closer together than the points of the first subset. In instances in which similar motion profiles are observed, similar distancing between points may be seen throughout the segment. However, the distancing between the points of the first and second subsets herein described differs, indicating that the motion profiles of the respective points differ.

The first and second strains 906, 908 within the first segment 904 may be considered together in the output of the speckle tracking echocardiography. The non-myocardium areas may thus effect the outputted strain for the first segment 904, resulting in non-physiological strain.

The segmented ROI 902 is again shown in FIG. 9C. Peak systolic strain of each segment of the segmented ROI 902 is indicated visually both textually within each segment and with shading within the ROI. The peak systolic strain of the first segment 904 may be statistically different from (e.g., larger than) the peak systolic strain of the other segments of the ROT. For example, the peak systolic strain of the first segment 904 may be −4% while the peak systolic strain of an adjacent second segment 918 of a plurality of segments 916 may be −18%. The outputted peak systolic strain for the first segment 904 may not accurately reflect the strain of the myocardium in the first segment 904. For example, the first strain 906 may be accurate to the strain of the myocardium while the second strain 908 may not be accurate to strain of the myocardium and may thus render the outputted strain for the segment inaccurate.

FIG. 9D shows a strain trace graph 930 demonstrating the statistical different between the strain of the first segment 904 and the strain of the other segments of the plurality of segments 916. The strain trace graph 930 may comprise a plurality of plots 932 indicating calculated strain (indicated by the y-axis) over time (over the course of a cardiac cycle, indicated by the x-axis). The plurality of plots 932 may comprise a plot for each of the segments of the segmented ROI 902. For example, a first plot 936 may plot the strain calculated for the first segment 904 and a plurality of second plots 934 may each plot the strain calculated for each of the other segments of the plurality of segments (e.g., for the second segment 918, a third segment, a fourth segment, a fifth segment, and a sixth segment).

Each of the plurality of second plots 934 may generally follow a similar path, with similar peak systolic strains, as denoted by indicators plotted at or near the nadir of each plot. The first plot 936, however, may not follow the similar path, again indicating the presence of non-physiological strain source(s) within the first segment 904.

FIG. 9E shows a 2D plane 950. The 2D plane 950 may plot the first subset 911 of the plurality of points 914 of the segmented ROI 902 according to the motion vector thereof. For example, the motion vector of each point may be transformed into the polar domain based on magnitude and direction (e.g., length and angle). The first subset 910 may have motion vectors that lie within a first section 952 of the 2D plane 950, while the second subset 912 may have motion vectors that lie within a second section 954 of the 2D plane 950. The first subset 910 may thus form a first motion cluster and the second subset 912 of points may form a second motion cluster that is different from the first motion cluster.

Identification of more than one motion cluster in a 2D transformation as is herein shown may indicate to the system that a source of non-physiological strain is present. Depending on the distribution of motion vectors within the 2D plane, the source of the non-physiological strain may be determined to be either ROI-based, as is presented in FIGS. 8A-8E and 9A-9E, or artifact-based, as will be described with respect to FIGS. 10A-C. When distinct clusters of motion vectors are identified, as shown in FIGS. 8E and 9E wherein two motion clusters are present, the source of non-physiological strain may be ROI-based. When diffuse motion vectors are identified without distinct clusters, the source of non-physiological strain may be artifact-based.

Turning now to FIGS. 10A-C, a cardiac ultrasound image 1000 including artifact-based non-physiological strain is shown. Starting with FIG. 10A, the cardiac ultrasound image 1000 is shown with an ROI 1002 overlaid thereon. As is described previously, the ROI 1002 may include a plurality of predefined segments, such as segment 1004. The ROI 1002 may comprise a plurality of points. Speckle tracking echocardiography may be applied to the points of the ROI 1002 in order to define strain over the course of a cardiac cycle based on motion of each point between contraction and expansion (e.g., between systole and diastole). The change in position of each point may define strain in percentage. Points 1006 of the segment 1004 may be tracked according to the speckle tracking algorithm. The points 1006 of the segment 1004, however, may represent or correspond to artifact or noise within the cardiac ultrasound image. When the points 1006 correspond to artifact, the corresponding motion field of the points may show that the points 1006 do not share any particular motion profile.

FIG. 10B shows a strain trace graph 1008. The strain trace graph 1008 includes a plot 1010 corresponding to the segment 1004. The plot 1010 may indicate calculated strain (e.g., on the y-axis) over time (e.g., on the x-axis) over the course of the cardiac cycle. In comparison to the strain trace plots described with respect to FIGS. 5 and 6, which do not have non-physiological strain, the plot 1010 may not provide diagnostically useful information. For example, the calculated strain may vary greatly over short intervals, thus indicating that the strain calculated is not accurate for the patient's myocardium.

The motion vectors of the points 1006 may be transformed into the polar domain in order to allow for determination of sources of non-physiological strain before calculating the strain values. As shown in FIG. 10C, a 2D plane 1050 of the polar domain may comprise a plurality of predefined sections 1052. Each of the points 1006 may be plotted within the 2D plane according to the motion vector of each point. As the points correspond to artifact or noise, the motion vectors may not be clustered together in any distinguishable fashion. Rather, the points may exhibit a motion pattern in which the points are diffusely scattered within the 2D plane 1050 about the origin.

A scattered motion pattern as is shown in FIG. 10C may indicate that the source of non-physiological strain is artifact-based rather than ROI-based. In comparison, the 2D planes of FIGS. 8E and 9E may show clearly distinct motion clusters rather than the scattered points of FIG. 10C, which may indicate that the source of non-physiological strain in those examples is ROI-based. Further, regions with reverb, haze, shadowing, or other artifact related to boney structures (e.g., ribs) typically do not have contractile tissue. Thus, without movement of their own, any identified motion may be motion that results from the motion of the contractile myocardial tissue. The motion vectors of the points that correspond to this type of artifact may thus be centered about the origin of the 2D plane, as shown in FIG. 10C.

When the source of non-physiological strain is ROI-based, the system as herein disclosed may correct the ROI in order to remove the source of non-physiological strain. Thus, the output from the speckle tracking echocardiography may not include non-physiological strain and may be more accurate, providing for more accurate diagnoses and saving time for the user as need to repeat studies or manually adjust the ROI may be reduced.

For example, FIGS. 11A and 11B show examples of ROI correction that correspond to the segmented ROIs 802 and 902, respectively, as described above. For example, starting with FIG. 11A, the first segment 804 of the segmented ROI 802 is shown. The first segment 804, as previously described, includes the first subset 812 of points and the second subset 814 of points. The first subset 812 may correspond to areas of myocardium and the second subset 814 may correspond to non-myocardium (e.g., pericardium, blood pooling, valve, etc.), as determined by the motion profiles thereof. The first subset 812 may be positioned laterally adjacent to the second subset 814, within the first segment 804.

Correction of ROI-based non-physiological strain when point sources (e.g., the second subset 814 of points) of non-physiological strain are positioned laterally next to accurately positioned points may include adjusting a width of the particular segment. For example, the second subset 814 of points may be removed from the segment.

In the example provided, upon correction (designated by an arrow in FIG. 11A) the second subset 814 is removed, and thus the first segment 804 is transformed into a corrected first segment 1104. The corrected first segment 1104 comprises only the first subset 812 of points. Thus, a corrected segmented ROI may be generated that does not contain any sources or non-physiological strain. In this way, the source of non-physiological strain may be removed and a resulting strain determined for the corrected first segment 1104 and the corrected segmented ROI may be physiological, as is further described below.

In FIG. 11B, the first segment 904 of the segmented ROI 902 is shown. The first segment 904, as previously described, includes the first subset 910 of points and the second subset 912 of points. The first subset 910 may correspond to areas of myocardium and the second subset 912 may correspond to non-myocardium (e.g., pericardium, blood pooling, valve, etc.), as determined by the motion profiles and resulting strain values thereof. The first subset 910 may be positioned below (e.g., vertically below with respect to a z-axis of the patient) the second subset 912, within the first segment 904.

Correction of ROI-based non-physiological strain when point sources (e.g., the second subset 912 of points) of non-physiological strain are positioned below accurately positioned points may include adjusting a height of the particular segment. For example, the second subset 912 of points may be removed from the segment.

In the example provided, upon correction (designated by an arrow in FIG. 11B) the second subset 912 is removed, and thus the first segment 904 is transformed into a corrected first segment 1106. The corrected first segment 1106 comprises only the first subset 910 of points. Thus, a corrected segmented ROI may be generated that does not contain any sources or non-physiological strain. In this way, the source of non-physiological strain may be removed and a resulting strain determined for the corrected first segment 1106 may be physiological, as is further described below.

In this way, the motion vectors of the points of the corrected first segment 1106 form a single cluster when transformed into the polar domain. A 2D plane plotted for a corrected ROI may thus comprise a single motion cluster as all of the points of the corresponding segment may share a motion profile.

FIG. 12A illustrates correction of the first segment 804 into the corrected first segment 1104. In a first display output 1200, the first segment 804 with the first and second subsets of points 812, 814 is shown. In a second display output 1202, the corrected first segment 1104 comprising only the first subset of points 812 is shown.

FIG. 12B shows strain trace graphs for each of the first and second display outputs 1200, 1202. For example, the strain trace graph 830 may correspond to the first display output 1200 that includes the uncorrected first segment 804. As explained with respect to FIG. 8D, which also displays the strain trace graph 830, the plurality of plots 832 displayed therein include the first plot 836 that corresponds to the uncorrected first segment 804 as well as one or more second plots 834 that correspond to the other segments of the segmented ROI 802. The strain of the uncorrected first segment 804 may be statistically different from the strain of the other segments, as indicated by the plots of the strain trace graph 830.

A corrected strain trace graph 1252 is also shown in FIG. 12B. The corrected strain trace graph 1252 may be outputted following ROI-based correction of non-physiological strain. The corrected strain trace graph 1252 may comprise a plurality of plots 1260, comprising a corrected first plot 1262 and the one or more second plots 834. The one or more second plots 834 may be unchanged from the strain trace graph 830 as ROI-based correction may affect segments that include sources of ROI-based non-physiological strain. The corrected first plot 1262 may correspond to the corrected first segment 1104. As a result, the corrected first plot 1262 may not be affected by sources of non-physiological strain and thus may follow generally a similar path to over the course of the cardiac cycle to the one or more second plots 834.

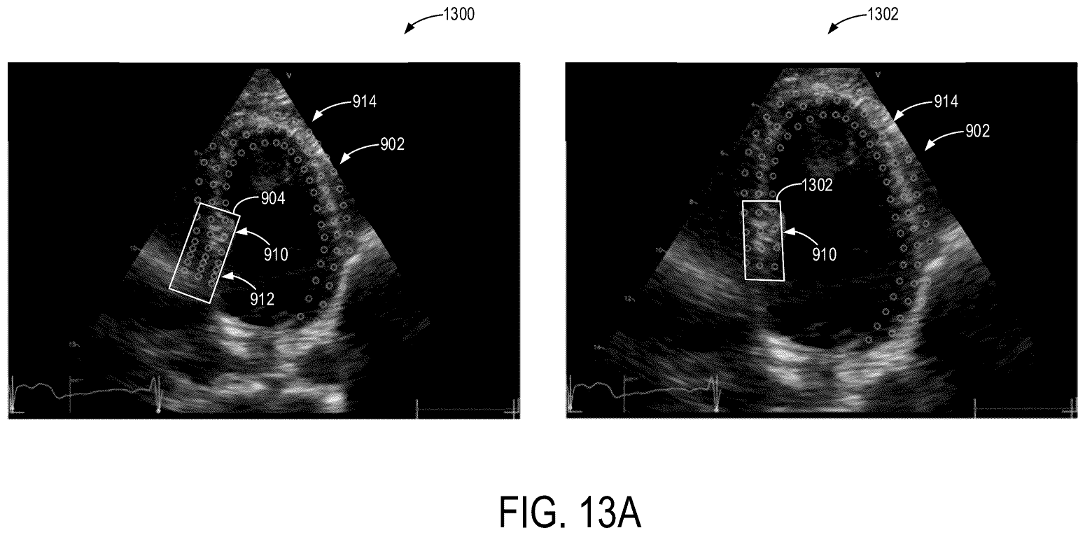

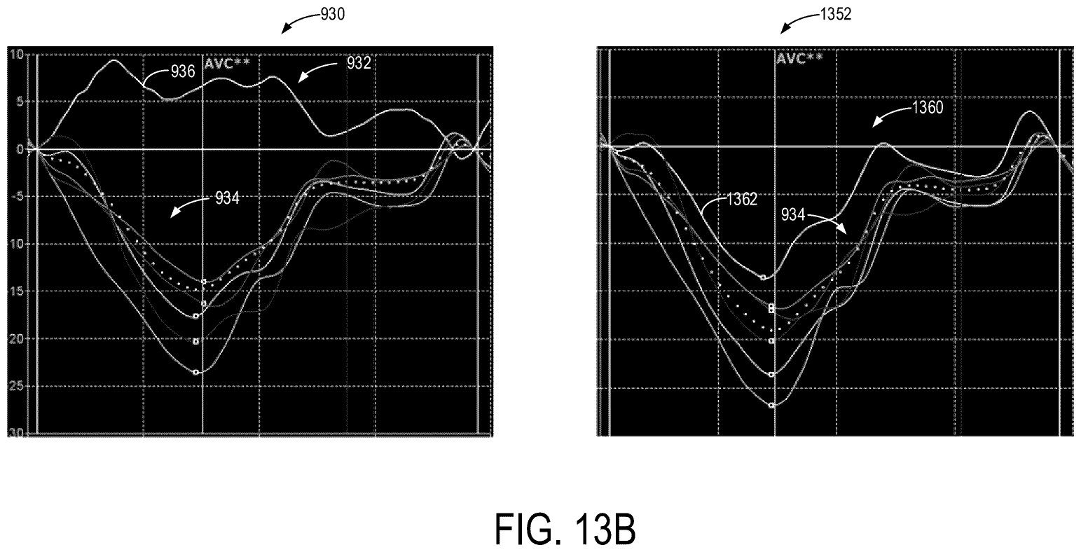

Similarly, FIG. 13A illustrates correction of the first segment 904 into the corrected first segment 1106. In a first display output 1300, the first segment 904 with the first and second subsets of points 910, 912 is shown. In a second display output 1302, the corrected first segment 1106 comprising only the first subset of points 910 is shown.

FIG. 13B shows strain trace graphs for each of the first and second display outputs 1300, 1302. For example, the strain trace graph 930 may correspond to the first display output 1300 that includes the uncorrected first segment 904. As explained with respect to FIG. 9D, which also displays the strain trace graph 930, the plurality of plots 932 displayed therein include the first plot 036 that corresponds to the uncorrected first segment 904 as well as one or more second plots 934 that correspond to the other segments of the segmented ROI 902. The strain of the uncorrected first segment 904 may be statistically different from the strain of the other segments, as indicated by the plots of the strain trace graph 930 due to non-physiological strain.

A corrected strain trace graph 1352 is also shown in FIG. 13B. The corrected strain trace graph 1352 may be outputted following ROI-based correction of non-physiological strain. The corrected strain trace graph 1352 may comprise a plurality of plots 1360, comprising a corrected first plot 1362 and the one or more second plots 934. The one or more second plots 934 may be unchanged from the strain trace graph 930 as ROI-based correction may affect segments that include sources of ROI-based non-physiological strain. The corrected first plot 1362 may correspond to the corrected first segment 1106. As a result, the corrected first plot 1362 may not be affected by sources of non-physiological strain and thus may follow generally a similar path to over the course of the cardiac cycle to the one or more second plots 934.

In this way, ROI-based correction may address non-physiological strain. By identifying and correcting points of the ROI that generate non-physiological strain, based on clustering of motion profiles as is herein described, points of the ROI that generate non-physiological strain may be removed. In doing so, the ROI may be corrected to remove the non-physiological strain calculated by speckle tracking echocardiography algorithms.

While the examples shown in the figures described above show ROI correction on an epicardial side of the myocardium, it should be appreciated that ROI correction may be applied on the endocardial border as well, for example in the instance of segmentation of an ROI that includes blood pool within the heart.

Turning to FIG. 14, a flowchart illustrating a method 1400 for ROI-based correction of non-physiological strain in speckle tracking echocardiography is shown. The method 1400 may be implemented for ultrasound images acquired by a suitable ultrasound imaging system, such as ultrasound imaging system 100 of FIG. 1, although other ultrasound imaging systems are feasible. The method 1400 may be implemented by a medical image processing system, such as medical image processing system 200 of FIG. 2. As such, the method 1400 may be stored as executable instructions in non-transitory memory, such as memory 120 of FIG. 1 and/or the non-transitory memory 206 of FIG. 2, and executed by a processor, such as the processor 116 of FIG. 1 and/or the processor 204 of FIG. 2. Further, in some embodiments, the method 1400 is performed in real-time, as the cardiac ultrasound images are acquired, while in other embodiments, at least portions of the method 1400 may be performed offline after the cardiac ultrasound images are acquired (e.g., following termination of a scan of a subject via the ultrasound imaging system, where, during the scan, a probe of the ultrasound imaging system may be energized to acquire cardiac ultrasound images of the subject, and following termination of the scan, the probe may not be energized to acquire cardiac ultrasound images). For example, the processor may evaluate cardiac ultrasound images that are stored in memory even while the ultrasound system is not actively being operated to acquire images.

At 1402, the method 1400 includes acquiring ultrasound imaging data of the heart. The ultrasound imaging data may be acquired according to an ultrasound protocol, which may be selected by an operator (e.g., user) of the ultrasound imaging system via a user interface (e.g., the user interface 115 of FIG. 1). As one example, the operator may select the ultrasound protocol from a plurality of possible ultrasound protocols using a drop-down menu or by selecting a virtual button. Alternatively, the system may automatically select the protocol based on data received from an electronic medical record (EMR) or radiology information system (RIS) record associated with the patient (e.g., for example, the patient may be scheduled for a particular protocol as stored in the EMR or RIS which may indicate which protocol the ultrasound system may run). Further, in some examples, the operator may manually input and/or update parameters to use for the ultrasound protocol. The ultrasound protocol may be a system guided protocol, wherein the system guides the operator through the protocol step-by-step, or a user guided protocol, where the operator follows a lab-defined or self-defined protocol without the system enforcing a specific set of steps or having prior knowledge of the protocol steps.

Further, the ultrasound protocol may include a plurality of views and/or imaging modes that are sequentially performed, in some examples. Using cardiac ultrasound imaging as an example as is herein presented, the ultrasound protocol may include a four-chamber view of the left ventricle with B-mode and a four-chamber view focused on the right ventricle with B-mode. Additionally or alternatively, the ultrasound protocol may include APLAX views of the heart, as is shown in FIG. 3. It may be understood that in some examples, a partial view of the heart may be acquired, such as a two-chamber view of the left ventricle and left atrium or a single chamber view (e.g., only the left ventricle). In some examples, additional imaging modes may be used, such as color flow imaging (CFI). Further, the ultrasound protocol may specify a frame-rate for acquiring the ultrasound imaging data. The frame-rate for the acquisition may be increased when a regional, partial view of the heart is acquired compared with a full acquisition because a field of view is smaller. In some examples, a plurality of regional views of the heart may be acquired, with each of the plurality of regional views obtaining a different partial view of the heart, in order to obtain more accurate mapping of strain values in each region. In the embodiment herein described, the protocol may be speckle tracking echocardiography.

The ultrasound imaging data may be acquired with an ultrasound probe by transmitting and receiving ultrasonic signals according to the ultrasound protocol. In the above cardiac ultrasound imaging example, performing the ultrasound protocol may include acquiring ultrasound data for some or all of the above-mentioned views and imaging modes. Acquiring the ultrasound data according to the ultrasound protocol may include the system displaying instructions on the user interface and/or display, for example, to guide the operator through the acquisition of the designated views. Additionally or alternatively, the ultrasound protocol may include instructions for the ultrasound system to automatically acquire some or all of the data or perform other functions. For example, the ultrasound protocol may include instructions for the user to move, rotate, and/or tilt the ultrasound probe, as well as to automatically initiate and/or terminate a scanning process and/or adjust imaging parameters of the ultrasound probe, such as ultrasound signal transmission parameters, or display parameters. Further, the acquired ultrasound data may include one or more image parameters calculated for each pixel or group of pixels (for example, a group of pixels assigned the same parameter value) to be displayed, where the one or more calculated image parameters include, for example, one or more of an intensity, texture, graininess, contractility, deformation, and rate of deformation value.

At 1404, the method 1400 includes generating cardiac ultrasound images from the acquired ultrasound imaging data. The cardiac ultrasound images may also be referred to herein as ultrasound images of the heart. At least one cardiac ultrasound image may be generated for each view of the ultrasound protocol. For example, the signal data acquired during the method at 1402 is processed and analyzed by the processor in order to produce an ultrasound image. The processor may include an image processing module that receives the signal data (e.g., imaging data) acquired at 1402 and processes the received imaging data. For example, the image processing module may process the ultrasound signals to generate slices or frames of ultrasound information (e.g., ultrasound images) for displaying to the operator. In one example, generating the image may include determining an intensity value for each pixel to be displayed based on the received imaging data (e.g., 2D or 3D ultrasound data). As such the generated cardiac ultrasound images may be 2D or 3D depending on the mode of ultrasound being used (e.g., CFI, acoustic radiation force imaging, B-mode, A-mode, M-mode, spectral Doppler, acoustic streaming, tissue Doppler module, C-scan, or elastography). The present example will be discussed for 2D B-mode cardiac ultrasound images, although it should be understood that any of the above mentioned modes or other imaging modes may be used according to the selected ultrasound protocol. In some examples, the generated cardiac ultrasound images may include multiple frames, according to the designated frame-rate, and thus may be considered motive images. For example, a cardiac ultrasound image may comprise multiple frames over the course of a cardiac cycle, as described previously.

At 1406, method 1400 includes generating a segmented ROI for a given cardiac ultrasound image. For example, the segmented ROI may be generated for a particular view, such as APLAX or four-chamber. The segmented ROI may be generated based on a combination of user inputs and a segmentation algorithm, in some examples. For example, the user may indicate via one or more mouse clicks, a plurality of edges of the ROI and the segmentation algorithm may generate boundaries of the ROI based on the designated edges. The segmented ROI may have predefined segments. For example, the ROI may be partitioned into six segments of equal area.

At 1408, method 1400 includes identifying speckle points within the segmented ROI. As one example, speckle tracking echocardiography protocols may include defining brighter pixel speckles (referred to herein as points or speckle points) depicted within the segmented ROI that are produced as a result of the scatter of the ultrasound beam by the tissue. The identified speckle points may each have motion vectors that are determinable, as will be described further with respect to FIG. 15.

At 1410, method 1400 includes identifying one or more sources of non-physiological strain. As is discussed above, non-physiological strain, when determined via speckle tracking, may result from inaccurately positioned ROI points or from artifact/noise. As is herein described, strain may be calculated for myocardium as the myocardium contracts and expands. Non-myocardium tissues or other interferences, such as pericardium, blood pooling, valvular structures, and the like may not be contractile tissue and thus may not move in the same manner as myocardial tissue. As a result, the strain calculated for speckle points that correspond to non-myocardium may not be accurate. Identifying the sources of non-physiological strain may comprise transforming motion vectors of each point of the segmented ROI into the polar or spherical domain in order to cluster by motion profile, as will be further described with respect to FIG. 15.

At 1412, method 1400 includes determining whether the one or more sources of non-physiological strain are ROI-based. As previously noted, the type of source may be either ROI-based or artifact-based. ROI-based sources may include misalignment of the ROI. Misalignment, as herein used, may include any positioning of the ROI that results in inclusion of pixels corresponding to non-myocardium. Artifact-based sources may include the identified speckle points corresponding to artifact, such as haze, noise, blur, etc., rather than myocardium or other heart tissue. Identification of sources of non-physiological strain may include determining whether motion profiles of pixels of a given segment are clustered into distinct groups or are scattered. When clustered into distinct groups, the type of source may be ROI-based. When the motion vectors are scattered without distinct groups and/or are generally centered about the origin, the type of source may be artifact-based. Alternatively or additionally, artifact-based sources may be identified through an algorithm aimed to identify distinctive patterns of motion, such as a machine learning or deep learning algorithms or a template matching algorithm. If the source is ROI-based (YES at 1412), method 1400 proceeds to 1414. If the source is not ROI-based, for example is artifact-based, (NO at 1412), method 1400 proceeds to 1420.

At 1420, method 1400 includes notifying the user of the artifact source. Notifying the user may include display of a notification on the user interface or display indicating that artifact, such as haze, reverberation, shadowing, or any other form of noise, is present and that an outputted strain trace may not be accurate. The notification may also include instructions to address the artifact, such as repeating the images, repeating the images with different probe angles, or the like. In some examples, the notification may include an option to correct the areas of artifact. In examples in which correction of artifact is not an option, method 1400 ends following 1420. In examples in which correction of artifact is an option, method 1400 proceeds to 1422.

At 1422, method 1400 optionally includes extrapolating motion for unreliable points. The unreliable points as herein described may be the points that correspond to the source of non-physiological strain. As will be described with respect to FIG. 15, unreliable points may be those points within the segmented ROI that do not have a similar motion profile to the majority of the points of the ROI (e.g., the reliable points). Extrapolating motion for unreliable points may include reducing weights of motion vectors obtained from the area with artifact and using the motion vectors of neighboring reliable segments to extrapolate the motion of the area with artifact. In some examples, extrapolation of motion for the area with artifact may be performed in response to user selection of a notification, for example when the notification as described at 1420 includes the option to correct the areas of artifact. In other examples, extrapolation of motion for the area with artifact may be performed automatically in response to detection of the artifact-based source of non-physiological strain. Method 1400 then proceeds to 1416 to apply a speckle tracking algorithm.

It should be understood that in some examples, more than one source of non-physiological strain may be identified within the image, for example within different segments of the ROI or within the same segment of the ROI. The more than one source may all be ROI-based, may all be artifact-based, or may be a combination thereof. In the instance of both ROI-based and artifact-based sources in the image, if any of the sources are artifact-based, method 1400 may proceed to 1418 to notify the user as explained above. If artifact-based correction is performed based on extrapolation of motion as described at 1422, the method 1400 would proceed to 1414 to then address the ROI-based sources. Alternatively, in examples in which correction for both ROI-based sources and artifact-based sources is available, correction may be performed simultaneously.

At 1414, in response to determination that the source is ROI-based, method 1400 includes auto-correcting the ROI. Auto-correcting the ROI may include removing the one or more points that correspond to non-myocardium, as is described with respect to FIGS. 11A-B. Auto-correcting the ROI may thus remove the source of non-physiological strain. The correction may result in a cluster of motion vectors that is not centered about an origin of a 2D plane within the polar domain (or 3D plane within the spherical domain), as described above. If the correction does not result in a single cluster, artifact may also be present, in which case the system may reject the ROI and a notification may be presented to the user and/or extrapolation may be performed as described above.

At 1416, method 1400 includes applying a speckle tracking algorithm to determine strain. The speckle tracking algorithm may be applied to the corrected ROI. As noted, the corrected ROI may not comprise sources of non-physiological strain. As described, ROI-based sources may be removed by correction of the ROI and/or artifact-bases sources may be removed by extrapolation as described above. The speckle tracking algorithm may be applied as part of a speckle tracking echocardiography protocol. As is described above, the ROI, which corresponds to myocardium, may be tracked throughout an entire cardiac cycle or a portion of the cardiac cycle to determine how each portion of the myocardium contracts and relaxes through the cardiac cycle. Each cardiac cycle comprises one heartbeat, which includes two periods called diastole and systole. During diastole, the myocardium relaxes and the heart fills with blood. During systole, the myocardium contracts to pump the blood out of the heart. The contraction and relaxation cause shortening and elongation of the myocardium, respectfully, which may be quantified via the speckle tracking algorithm and outputted as strain.

The speckle tracking algorithm may include a pre-programmed analysis tool or algorithm that calculates the strain at a given position of the ROI (e.g., within a segment of the segmented ROI) in the cardiac ultrasound images as a change in length of the heart muscle at the given position between two time points. The given position as herein noted may be a particular point of a segment. The strain at the given position may change throughout the cardiac cycle as the muscle expands and contracts. Further, strain values may be determined in a plurality of directions (e.g., longitudinal, circumferential, and radial) that correspond to the change in length in the corresponding direction. Each strain value may be given as a percentage change (e.g., negative or positive) between an initial time point (e.g., before an electrical pulse through the heart causes contraction) and a final time point (e.g., after the electrical pulse through the heart causes contraction), for example. Further, the speckle tracking algorithm may generate one or both of regional strain values corresponding to individual segments of the segmented ROI and global strain values corresponding to an entirety of the segmented ROI, in some examples.

At 1418, method 1400 includes outputting a strain trace graph and annotated echocardiography images to the display device. The annotated echocardiography images may include overlays of the segmented ROI, shading based on calculated strain, and/or textual representations of the calculated strain, such as is depicted in FIGS. 8C and 9C. The strain trace graph, as described with respect to FIGS. 8D and 9D, may plot strain (in percent value) over time for the course of the cardiac cycle. In some examples, the strain trace plot may display strain values for each segment of the segmented ROI. In other examples, the strain trace plot my display a global strain values for the entire segmented ROI.

The methods herein for ROI-based correction of non-physiological strain may reduce overall processing power demands for the computing device as compared to other techniques, which may include entirely repeating the segmentation of the ROI and calculating strain via application of a speckle tracking algorithm, which both can have high processing demands, or repeating the scan acquisition all together. In this way, more accurate strain values may be achieved while reducing the overall processing demands on the system in the presence of non-physiological strain.

Turning now to FIG. 15, a flowchart illustrating a method 1500 for identifying sources of non-physiological strain is shown. The method 1500 may be implemented for ultrasound images acquired by a suitable ultrasound imaging system, such as ultrasound imaging system 100 of FIG. 1, although other ultrasound imaging systems are feasible. The method 1500 may be implemented by a medical image processing system, such as medical image processing system 200 of FIG. 2. As such, the method 1500 may be stored as executable instructions in non-transitory memory, such as memory 120 of FIG. 1 and/or the non-transitory memory 206 of FIG. 2, and executed by a processor, such as the processor 116 of FIG. 1 and/or the processor 204 of FIG. 2. Further, in some embodiments, the method 1500 is performed in real-time, as the cardiac ultrasound images are acquired, while in other embodiments, at least portions of the method 1500 may be performed offline after the cardiac ultrasound images are acquired (e.g., following termination of a scan of a subject via the ultrasound imaging system, where, during the scan, a probe of the ultrasound imaging system may be energized to acquire cardiac ultrasound images of the subject, and following termination of the scan, the probe may not be energized to acquire cardiac ultrasound images). For example, the processor may evaluate cardiac ultrasound images that are stored in memory even while the ultrasound system is not actively being operated to acquire images.

Method 1500 may be implemented as part of a speckle tracking echocardiography protocol, for example at 1408 in method 1400 described above. As such, cardiac ultrasound data may be acquired, cardiac ultrasound images may be acquired, and speckle points with motion data thereof of the segmented ROI may be identified prior to method 1500.

At 1502, method 1500 includes transforming motion data of the speckle points into the polar domain for 2D applications (or spherical domain for 3D applications). In some examples, speckle points of each segment of the segmented ROI may be considered separately when speckle tracking is performed for each segment individually. In other examples, speckle points for the entire ROI may be considered together when speckle tracking is performed globally. Transforming the motion data of the speckle points may include determining a motion vector of each point, as noted at 1504. The motion vector of each point may include a magnitude and direction (e.g., a length and angle). This motion vector may then be transformed into the polar domain (e.g., transformed into polar coordinates) based on the magnitude and direction.

As an example, a displacement vector in Cartesian coordinates may be (Δx, Δy). These Cartesian coordinates may be converted to polar coordinates (r, θ) using equations (1) and (2):

$$r = \sqrt{Δ {x}^{2} + Δ {y}^{2}} \qquad ( 1 )$$

$$θ = {tan}^{- 1} 2 ( Δ y , Δ x ) \qquad ( 2 )$$

where r is magnitude of displacement and θ is angle from positive x-axis. Similarly, a displacement vector in Cartesian coordinates may be (Δx, Δy, Δz). These Cartesian coordinates may be converted to spherical coordinates (ρ, θ, φ) using equations (3), (4), and (5):

$$ρ = \sqrt{Δ {x}^{2} + Δ {y}^{2} + Δ {z}^{2}} \qquad ( 3 )$$

$$θ = {tan}^{- 1} 2 ( Δ y , Δ x ) \qquad ( 4 )$$

$$φ = {cos}^{- 1} Δ z / ρ \qquad ( 5 )$$

where ρ is the magnitude of displacement and φ is the polar angle from the z-axis.

At 1506, method 1500 includes generating a 2D (or 3D) plane in the polar (or spherical) domain that is divided into a plurality of sections. In some examples, as is depicted in FIGS. 7A-7B, 8E, and 9E, 2D planes may be generated for each segment. Each 2D plane may be partitioned into a plurality of sections via polar domain discretization based on radial and angular steps. The section parameters, including number of sections and size of each section, may be predefined in the system, in some examples. In other examples, the section parameters may be defined by the user or based on protocol parameters for the speckle tracking echocardiography protocol.

At 1508, method 1500 includes plotting each point within the 2D plane based on its corresponding motion vector. Plotting each point within the 2D plane may comprise plotting each point within a particular section of the 2D plane based on the motion vector. With displacement vectors defined by angles and radii, the vectors can be mapped to specific subregions of the planes. In examples in which 2D planes are generated for each segment, points of each segment may be assigned to sections of a corresponding 2D plane.

At 1510, method 1500 includes determining, based on the assignment of each point, motion clusters of points. When 2D planes are generated for each segment of the segmented ROI and points of a given segment are plotted within a corresponding 2D plane based on their motion vectors, one or more motion clusters may be determined for that given segment. Motion clusters may be defined as a group of points with motion vectors in the same section of the 2D plane. Points in the same motion cluster may thus share a motion profile.

When two or more motion clusters are determined within a 2D plane, at least one source of non-physiological strain may be identified. At 1512, method 1500 includes identifying any unreliable points of each segment. The unreliable points may correspond to source(s) of non-physiological strain. As an example, for an ROI-based source of non-physiological strain, a first motion cluster may be determined within a first segment and a second motion cluster may be determined within a second segment. One of the first motion cluster and the second motion cluster may correspond to unreliable points and the other of the first and second motion clusters may correspond to unreliable points. The reliable points may correspond to myocardium and the unreliable points may correspond to non-myocardium. The reliable points may be determined based on motion profile with respect to other segments. For example, if a neighboring segment's 2D plane comprises only a single motion cluster, that motion cluster may be considered reliable. The reliable motion cluster may then be used for comparison to determine which of the first and second motion clusters are reliable, in some examples.

Determining reliable and unreliable points from multiple motion clusters may be based on relative size of each cluster. For example, larger clusters may be generally considered more reliable, following a majority voting principle. For instance, in a sample of 20, a cluster with 1-2 samples may be deemed unreliable, while another cluster comprising 50% or more of the sample may be deemed unreliable. In the case of myocardium motion analysis for strain calculation, motion vectors with displacements larger than zero and belonging to sizeable clusters (e.g., containing 50% of samples) may be considered reliable.

In this way, by clustering motion vectors of speckle points within the segmented ROI, outlying, unreliable points may be identified. The unreliable speckle points may thus be considered as sources of non-physiological strain and those points may be corrected for as is described with respect to method 1400. The motion patterns, for example where the motion vectors are in the 2D plane with respect to the origin and/or how many clusters are present, may inform as to which type of source, ROI-based or artifact-based, is present. Thus, by transforming motion vectors of speckle points into the polar or spherical domain and identifying sources of non-physiological strain in order to correct for those sources, accuracy of speckle tracking and outputted strain values may be increased.

Further, by identifying sources of non-physiological strain prior to outputting strain values, for example during speckle tracking, the system may more efficiently correct for such sources. As described above, even if an outlying strain trace indicative of non-physiological strain was able to be identified by the user, in order to correct for it, segmentation of the ROI and calculation of strain via application of a speckle tracking algorithm may need to be repeated in its entirety in order to achieve an ROI that does not include sources of non-physiological strain, in the case of ROI-based sources. In the case of artifact-based sources, scan acquisition may also need to be repeated, in which case all the steps of strain calculation including ROI segmentation are performed again. Both repeating the segmentation and strain calculation and repeating the scan acquisition may be costly from a processing standpoint and may be time consuming for the user. Thus, incorporating identification of non-physiological strain source and correction thereof into the process of determining strain may increase efficiency of the system overall.

The methods herein described may also be applied to detection of other clinical parameters derived from strain traces, such as strain rate. Thus, the methods and systems herein disclosed may also be applied to other cardiac parameters calculated via speckle tracking echocardiography.

The technical effect of the systems and methods for identification and correction of non-physiological strain traces as herein presented is that accuracy and strain calculation may be increased. For example, by determining motion vectors of speckle points and identifying motion clusters thereof, sources of non-physiological strain, both from ROI misalignment and from artifact, may be identified and automatically corrected in a more accurate way. In this way, sources of non-physiological strain may be removed or otherwise accounted for, thereby reducing inclusion of non-physiological strain sources in the calculation of strain and increasing accuracy of strain calculations and thus resulting generated images. Increased accuracy of strain calculations may also increase diagnostic usability of the strain values and outputs as well as reducing time spent by the user in analyzing images and decreasing need for repeated image acquisitions.

As used herein, the terms “system” and “module” may include a hardware and/or software system that operates to perform one or more functions. For example, a module or system may include or may be included in a computer processor, controller, or other logic-based device that performs operations based on instructions stored on a tangible and non-transitory computer readable storage medium, such as a computer memory. Alternatively, a module or system may include a hard-wired device that performs operations based on hard-wired logic of the device. Various modules or systems shown in the attached figures may represent the hardware that operates based on software or hardwired instructions, the software that directs hardware to perform the operations, or a combination thereof.

“Systems” or “modules” may include or represent hardware and associated instructions (e.g., software stored on a tangible and non-transitory computer readable storage medium, such as a computer hard drive, ROM, RAM, or the like) that perform one or more operations described herein. The hardware may include electronic circuits that include and/or are connected to one or more logic-based devices, such as microprocessors, processors, controllers, or the like. These devices may be off-the-shelf devices that are appropriately programmed or instructed to perform operations described herein from the instructions described above. Additionally or alternatively, one or more of these devices may be hard-wired with logic circuits to perform these operations.

1. A system, comprising:
an ultrasound probe comprising at least one transducer, a matching layer, and a damping block;
a display device; and
a processor configured to execute instructions stored in non-transitory memory that, when executed, cause the processor to:
acquire ultrasound imaging data of a heart via the ultrasound probe;
generate cardiac ultrasound images from the acquired ultrasound imaging data of the heart;
generate a region of interest (ROI) comprising a plurality of segments;
identify a plurality of speckle points within each of the plurality of segments of the ROI;
determine motion vectors of each of the plurality of speckle points within each of the plurality of segments;
determine, based on the motion vectors, one or more sources of non-physiological strain corresponding to one or more of the plurality of speckle points;
identify a type of the one or more sources of non-physiological strain;
in response to identifying the type as ROI-based, correct the ROI to generate a corrected ROI;
determine cardiac strain values in the corrected ROI via a speckle tracking algorithm; and
output the cardiac strain values on the display device.

2. The system of claim 1, wherein to determine, based on the motion vectors, one or more sources of non-physiological strain, the processor is configured to execute further instructions stored in the non-transitory memory that, when executed, cause the processor to:
transform the motion vectors into the polar domain;
plot transformed motion vectors of each segment of the ROI in a respective 2D plane, wherein each respective 2D plane is partitioned into a plurality of predefined sections; and
determine one or more clusters of motion vectors for each segment based on position of the motion vectors within a corresponding 2D plane.

3. The system of claim 1, wherein, to correct the ROI, the processor is configured to execute further instructions stored in the non-transitory memory that, when executed, cause the processor to, in response to identifying the type as ROI-based, remove the one or more of the plurality of speckle points from the ROI to generate the corrected ROI.

4. The system of claim 1, wherein the processor is further configured to execute further instructions stored in the non-transitory memory that, when executed, cause the processor to, in response to identifying the type as artifact-based, extrapolate motion of the one or more of the plurality of speckle points from neighboring speckle points to generate the corrected ROI.

5. The system of claim 1, wherein the processor is further configured to execute further instructions stored in the non-transitory memory that, when executed, cause the processor to, in response to identifying the type as artifact-based, output a notification to a user via the display device that the one or more sources of non-physiological strain are present.

6. The system of claim 1, wherein to identify the type of source, the processor is configured to execute further instructions stored in the non-transitory memory that, when executed, cause the processor to identify motion patterns of the motion vectors within each segment, wherein, the type of source is ROI-based when the motion patterns include motion vectors clustered into distinct groups and the type of source is artifact-based when the motion patterns include scattered motion vectors centered around an origin of the respective 2D planes.

7. An ultrasound system, comprising:
an ultrasound probe configured to acquire cardiac ultrasound images; and
a computing device comprising one or more processors configured to execute instructions stored in non-transitory memory that, when executed, cause the computing device to:
generate cardiac ultrasound images from ultrasound imaging data of a heart;
generate a segmented region of interest (ROI) of the cardiac ultrasound images;
identify a plurality of points within the segmented ROI;
determine one or more of the plurality of points that correspond to one or more sources of non-physiological strain;
correct for the one or more of the plurality of points that correspond to the one or more sources of non-physiological strain to generate a corrected segmented ROI with a corrected plurality of points; and
calculate strain values for the corrected segmented ROI.

8. The ultrasound system of claim 7, wherein identifying the one or more of the plurality of points that correspond to the one or more sources of non-physiological strain comprises:
determining motion vectors of the plurality of points; and
determining one or more motion clusters based on the motion vectors.

9. The ultrasound system of claim 7, wherein the computing device is further configured to identify a type of source of non-physiological strain, wherein the type of source of non-physiological strain is one of ROI-based non-physiological strain and artifact-based non-physiological strain.

10. The ultrasound system of claim 9, wherein, when the type of source of non-physiological strain is ROI-based non-physiological strain, correcting for the one or more sources of non-physiological strain comprises correcting the segmented ROI by removing the one or more of the plurality of points that correspond to one or more sources of non-physiological strain.

11. The ultrasound system of claim 9, wherein, when the type of source of non-physiological strain is artifact-based non-physiological strain, correcting for the one or more sources of non-physiological strain comprises extrapolating motion for the one or more of the plurality of points that correspond to the one or more sources of non-physiological strain.

12. The ultrasound system of claim 7, wherein calculating strain values comprises applying a speckle tracking algorithm to the corrected segmented ROI.

13. The ultrasound system of claim 12, wherein calculating the strain values via the speckle tracking algorithm comprises determining a positional change in each of the corrected plurality of points of the corrected segmented ROI between consecutive image frames of the cardiac ultrasound images.

14. The ultrasound system of claim 7, further comprising outputting a strain trace graph comprising a plurality of plots plotting strain over a course of a cardiac cycle imaged in the cardiac ultrasound images, each of the plurality of plots corresponding to one of a plurality of segments of the corrected segmented ROI.

15. The method of claim 7, wherein the corrected segmented ROI corresponds to myocardium and the one or more of the plurality of points that correspond to the one or more sources of non-physiological strain correspond to non-myocardium.

16. The method of claim 7, wherein the segmented ROI is generated via one or more of user inputs and one or more segmentation algorithms applied to the cardiac ultrasound images.

17. A system, comprising:
an ultrasound imaging system comprising an ultrasound probe, a display device, and a computing device comprising memory storing instructions executable by a processor that when executed cause the processor to:
generate cardiac ultrasound images from ultrasound imaging data of a heart, wherein the cardiac ultrasound images comprise a plurality of frames throughout a cardiac cycle;
determine a segmented region of interest (ROI) within the cardiac ultrasound images, wherein the segmented ROI comprises a plurality of segments;
identify a plurality of speckle points within the segmented ROI, wherein each segment of the segmented ROI comprises a subset of the plurality of speckle points;
determine, for each speckle point in each subset of the plurality of speckle points, a motion vector;
transform the motion vector of each speckle point of each subset of the plurality of speckle points to a polar domain;
for each segment, determine one or more motion clusters of motion vectors;
for each segment, identify one or more of the motion clusters as unreliable, wherein speckle points corresponding to the one or more of the motion clusters identified as unreliable are sources of non-physiological strain;
for each segment, determine a type of the sources of non-physiological strain;
for each segment, in response to determination of the type as ROI-based, remove the speckle points corresponding to the one or more of the motion clusters identified as unreliable from the segmented ROI;
apply a speckle tracking algorithm to the segmented ROI without the one or more of the motion clusters identified as unreliable to calculate strain; and
output the strain to the display device.

18. The system of claim 17, wherein outputting the strain to the display device comprises generating a strain trace graph comprising a plot for each segment of the segmented ROI plotting strain over the cardiac cycle.

19. The system of claim 17, wherein outputting the strain to the display device comprises generating an annotated cardiac ultrasound image displaying visual and textual representations of the strain for each segment.

20. The system of claim 17, wherein the computing device is further equipped with instructions that when executed cause the processor to, in response to determination of the type as artifact-based, extrapolate motion for the speckle points corresponding to the one or more of the motion clusters identified as unreliable from neighboring speckle points.
